(19) United States
(12) Patent Application Publication
(10) Pub. No.: US 2017/0326381 A1
(43) Pub. Date: Nov. 16, 2017

(54) WIRELESS MICRO/NANO- STIMULATION OPTO-ELECTRODE FOR EXCITABLE TISSUE

(71) Applicant: University of Pittsburgh - Of the Commonwealth System of Higher Education, Pittsburgh, PA (US)

(72) Inventors: Takashi Daniel Yoshida Kozai, Pittsburgh, PA (US); Alberto Luis Vazquez, Pittsburgh, PA (US)

(73) Assignee: University of Pittsburgh - Of the Commonwealth System of Higher Education, Pittsburgh, PA (US)

(21) Appl. No.: 15/526,135

(22) PCT Filed: Nov. 11, 2015
(86) PCT No.: PCT/US2015/060070

§ 371 Date: May 11, 2017

Related U.S. Application Data
(60) Provisional application No. 62/078,779, filed on Nov. 12, 2014.

(51) Int. Cl. A61N 5/06; A61N 1/05

(52) U.S. Cl. CPC A61N 5/0622; A61N 1/0541; A61N 1/0531; A61N 2005/0662; A61N 5/0601

(57) ABSTRACT

Disclosed are wireless stimulation electrode for excitable tissue. In one example, a wireless stimulation electrode includes a body made of biocompatible photovoltaic and/or photothermal material; and at least one coating surrounding at least a portion of the body, wherein at least one critical dimension of the body is substantially smaller than a target cell. Also disclosed are systems and methods for stimulating excitable tissue.

### CROSS REFERENCE TO RELATED APPLICATION

This application claims priority to and the benefit of U.S. Provisional Application No. 62/078,779, filed Nov. 12, 2014, which is hereby incorporated by reference in its entirety.

### ACKNOWLEDGMENT OF GOVERNMENT SUPPORT

This invention was made with government support under Grant Nos. NS062019 and NS066131 awarded by the National Institutes of Health. The government has certain rights in the invention.

### FIELD

This disclosure relates to the field of electrodes, such as wireless micro/nano-stimulation electrodes for excitable tissue and methods of use thereof.

### BACKGROUND

Electrical neural stimulation and modulation is important in many basic science applications as well as many clinical applications. Some of the approved electrical stimulators include artificial cardiac pacemaker, cochlear implants, and deep brain stimulation electrodes. A challenge with this technology is achieving a highly localized stimulated area (i.e., a small population of neurons). While microelectrodes enable high selectivity, these smaller electrodes can cause tissue damage during electrical stimulation due to higher stimulation current densities. Reactive tissue reaction from mechanical mismatch also leads to cellular encapsulation of the electrodes. Glial scar can form around the electrodes increasing the electrical impedance. In turn, electrical stimulation loses its effectiveness to excite electrically excitable tissue without increasing stimulation currents. Increasing the electrical stimulation current beyond a certain threshold can cause injury to the tissue and/or electrode causing permanent damage to the patient. Further, current electrical stimulation implants are tethered typically to the skull or other bones. This increases the impact of mechanical mismatch induced inflammation and immune tissue reaction as well as increase the probability of traumatic impact-induced mechanical failure of the implants. Thus, there is a need in the art to develop electrodes which allow highly localized stimulation without causing tissue damage.

### SUMMARY OF THE DISCLOSURE

Disclosed herein is an innovative non-wired non-tethered, free-floating implantable electrical stimulation micro/nano-electrodes. Implantable infrared (IR) lasers or LEDs (with or without implantable optic wave guides) or lasers/LEDs placed on the surface of the skin or subcutaneously are used to transmit photons. Red, far-red, and infrared light have longer wavelength, and can penetrate the tissue much deeper than visible light. These Red-to-IR photons are absorbed onto fully implanted photoelectric converters or antennas. The photoelectric and/or photothermal converters (herein photoconverter) are an electrical conductor or semi-conductor which can be further enhanced with a dopant as well as coatings to absorb and filter different wavelengths. Relatively high power sources such as lasers or diodes can be used to deliver photons which can be converted to electrical current and/or thermal gradient via photo-converters in single-photon or multiphoton events. This current and/or thermal gradient can then be guided and discharged into electrically excitable tissues, such as but not limited to neurons and muscle cells.

In some embodiments, a wireless stimulation electrode comprises a body made of biocompatible photovoltaic and/or photothermal material; and with or without one or more coatings surrounding at least a portion of the body, wherein at least one critical dimension of the body is substantially smaller than a target cell. In some embodiments, one or more of the coatings may be conductive coatings with drug reservoirs can further be applied onto the photo-converters. Photon excited photo-converters can then actuate the drug reservoir coating to release drugs. Lastly, the conductive coatings can be molecularly imprinted to scavange naturally produced molecules or proteins for rechargeable, photo-activation controlled drug release.

Also disclosed are methods for stimulating excitable tissue. In some embodiments, a method for stimulating excitable tissue comprises introducing at least one biocompatible photovoltaic and/or photothermal device into target excitable tissue and focusing photons, two-photons, biphotons, or multiphotons with wavelength between 400-2,000 nanometers onto at least one photovoltaic and/or photothermal device, wherein the photovoltaic and/or photothermal device has one critical dimension smaller than a cell of the target excitable tissue.

Systems for wirelessly stimulating a neuron are also provided. In some embodiments, a system comprises a subcellular sized biocompatible photovoltaic and/or photothermal device for stimulating excitable tissue, a coherent light source wirelessly coupled to the subcellular sized photovoltaic and/or photothermal device for stimulating excitable tissue, and if desired a focusing device adapted to focus light from the coherent light source onto the subcellular sized photovoltaic and/or photothermal device, thereby providing a system for wirelessly stimulating a neuron.

Systems and methods for wireless drug release are also provided.

The foregoing and other features and advantages of the disclosure will become more apparent from the following detailed description of several embodiments which proceeds with reference to the accompanying figures.

### BRIEF DESCRIPTION OF THE FIGURES

FIG. 1 shows a schematic of an exemplary wireless electrode illustrating the photovoltaic body surrounded by an optically selective coating and a biocompatible dielectric.

FIG. 2 shows a cross section of an exemplary embodiment of a wireless electrode incorporating nanostructure protrusions to facilitate endocytosis.

FIG. 3 shows a cross section of an exemplary embodiment of a wireless electrode with biocompatible dielectric coating functionalized with a bioactive coating.

FIGS. 4A-4D show exemplary wireless electrodes with and without coatings and incorporating an integrated waveguide.

FIG. 5 shows various embodiments of a wireless electrode interacting with a neuron.

FIG. 6 shows penetration of light into tissue as a function of both the optical scattering coefficient and the absorption coefficient.

FIG. 7 shows an example system for stimulating an electrode with integrated waveguide from a light source.

FIGS. 8A and 8B show example systems with a wireless electrode implanted in a brain and laser light directed through a deflectable micro-waveguide to stimulate neuronal tissue.

FIG. 9 shows an example system with a wireless electrode implanted in a brain and laser light directed from a VCSEL laser array to stimulate neuronal tissue.

FIGS. 10A and 10B show two-photon images obtained during stimulation of brain tissue using an untethered photoelectric electrode (A) and two-photon images of control glass pipettes (B).

FIG. 11 illustrates mask (black) applied to two-photon rastering laser prevents photoelectric activation on the electrode.

FIG. 12 shows fluorescent intensity of calcium indicator dye labeled neurons next to the photovoltaic stimulation electrode.

FIG. 13 illustrates untethered (open-circuit) 7 μm diameter parylene-C insulated carbon fiber Photovoltaic Electrode (dashed line) stimulated with 900 nm laser at 150 Hz and 10 mW excited GCaMP3 neurons in mouse brain. Activated neurons are indicated by the arrow.

FIGS. 14A-14D show examples of qualitative and quantitative results around a nonfunctional edge lattice probe. FIG. 14A illustrates GFAP and OX42 antibodies labeled astrocytes and microglia, respectively. FIG. 14B shows GFAP and NeuN labeled astrocytes and neuronal nuclei. FIG. 14C is a graph of normalized mean non-neuronal density as a function of distance from probe interface. FIG. 14D is a graph of mean neuronal density. P<0.05. Scale=100 μm

FIGS. 15A-15C show a 7 μm diameter composite microthread electrode consisting of a carbon-fiber core, a parylene-based thin-film dielectric insulator that is chemically functionalized to control intrinsic biological processes, and a PEDOT/PSS recording pad that has been demonstrated to record electrophysiological signal more robustly than traditional electrodes. FIG. 15A is a SEM image of an exemplary microthread electrode (MTE). FIG. 15B is a graph illustrating single-unit yield of MTE (upper two tracings) versus Silicon Mi Probe (lower two tracings). FIG. 15C is a graph of single-unit SNR of MTE (top trace) versus Silicon Mi Probe (bottom trace). Chronic neural spike activity was reported as it is considered to be one of the most sensitive assays of chronic neural interface.

FIGS. 16A and 16B show neurons binding to 4 week chronic implants of L1 Cell Adhesion Molecule (L1CAM) coated probes (FIG. 16A) and uncoated probes (FIG. 16B) showing no neural binding (B).

FIGS. 17A and 17B show photovoltaic stimulation of carbon fiber (FIG. 17A) and n-doped silicon (FIG. 17B).

FIGS. 18A-18D show examples of monolithically integrated micro-waveguides.

FIGS. 19A and 19B show a schematic (FIG. 19A) and an array (FIG. 19B) of a vertical cavity surface emitting lasers (VCSEL).

FIGS. 20A and 20B illustrates an in vivo longitudinal biocompatibility and stability comparison of disclosed photovoltaic and/or photothermal microelectrodes to state-of-the-art stimulation electrodes. FIG. 20A is a schematic of a chronic window preparation with stimulating electrode or optical fiber in M1 in which images are collected over M1 and S1. FIG. 20B is a two-photon image through chronic window of a 3 month chronically implanted electrode in a microglia-GFP and red intravascular dye (SR101) labeled cortex.

FIG. 21 is an image of a coronal section of the mouse cortex expressing Channelrhodopsin-2 achieved by means such virus transduction (e.g. AAV-hSyn-ChR2).

FIG. 22 is an illustration of an exemplary wireless drug delivery system in use.

FIG. 23 is a graph illustrating wireless drug release.

FIG. 24 is a bar graph illustrating wireless drug release.

### DETAILED DESCRIPTION OF SEVERAL EMBODIMENTS

The disclosure is set forth below in the context of multiple representative embodiments, which are not intended to be limiting in any way.

The drawings are intended to illustrate the general manner of construction and are not necessarily to scale. In the detailed description and in the drawings themselves, specific illustrative examples are shown and described herein in detail. It will be understood, however, that the drawings and the detailed description are not intended to limit the invention to the particular forms disclosed, but are merely illustrative and intended to teach one of ordinary skill how to make and/or use the invention claimed herein.

As used in this application and in the claims, the singular forms “a,” “an,” and “the” include the plural forms unless the context clearly dictates otherwise. Additionally, the term “includes” means “comprises.” Further, the term “coupled” encompasses mechanical as well as other practical ways of coupling or linking items together, and does not exclude the presence of intermediate elements between the coupled items.

The described things and methods described herein should not be construed as being limiting in any way. Instead, this disclosure is directed toward all novel and non-obvious features and aspects of the various disclosed embodiments, alone and in various combinations and sub-combinations with one another. The disclosed things and methods are not limited to any specific aspect or feature or combinations thereof, nor do the disclosed things and methods require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed devices and methods can be used in conjunction with other devices, systems and method. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In the following description, certain terms may be used such as “up,” “down,”, “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

I. Introduction

Implantable microscale neural stimulation electrodes are an enabling technology for neuroscience. Since Italian physician Luigi Alyisio Galvani discovered that the muscles of a dead frog leg twitched when an electrical spark stuck an exposed nerve in the 1770's, researchers have been using electrical stimulation to study the nervous system. Electrical stimulation has been applied to alter behavior and learning, study memory and plasticity, and interrogate neural circuitry and network connectivity.

Current Challenges and Limitations

Electrical Stimulation. A challenge with electrical stimulation is the tradeoff between safety limits and spatial selectivity of the stimulated neural population. Stimulation electrodes with small surface areas can result in high charge densities that lead to permanent damage of the electrode or permanent damage to the tissue and nearby neurons. Therefore, large stimulation electrodes are frequently used, which leads to a spatially broad orthodromic and antidromic activation of neurons. In addition, electrodes tethered to the skull have increased probability of mechanical failure, ranging from mechanical breakage to insulation failure. Tethering electrodes further enhance mechanical mismatch, inducing strain that aggravates reactive tissue reaction, glial scarring, and neuronal loss. As a result, greater charge density stimuli for efficacious activation of neurons are needed, which can potentially lead to further damage and higher power consumption. Furthermore, the mechanical strain can result in positional drifts and movement of the electrode which may be detrimental for probing fine changes in neural circuits longitudinally (e.g., plasticity).

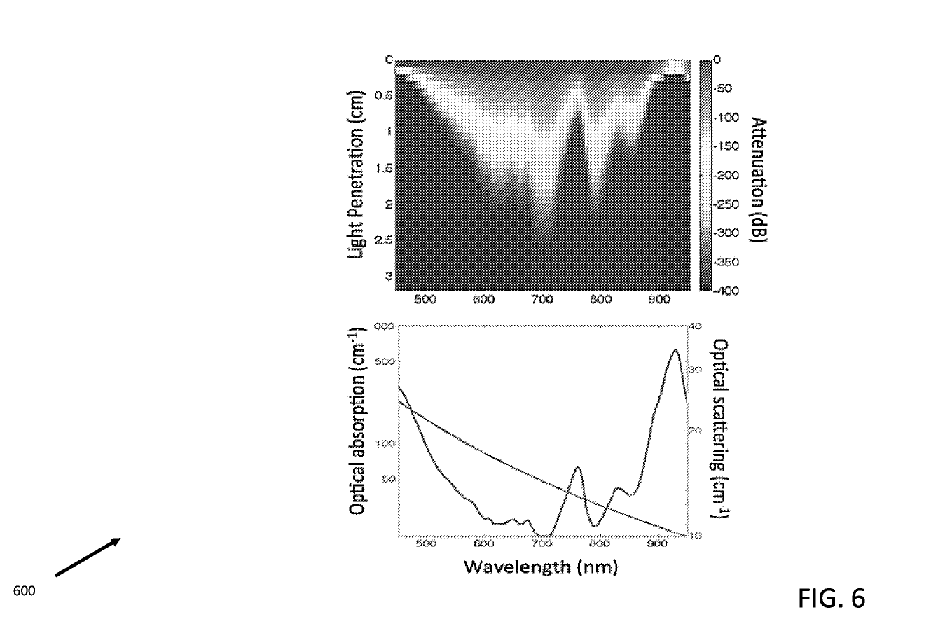

With reference to FIG. 6, plot 600 shows light absorption and penetration in tissue. The penetration of light into tissue is a function of both the optical scattering coefficient and the absorption coefficient. At UV and visible colors (200-550 nm), both optical absorption and scattering is high and light is highly attenuated. In the so-called “near-IR window” between 550-900 nm and “IR window” between 1,300 nm and 1,700 nm, the optical absorption coefficient is low enough, such as about 0.05 mm⁻¹, to allow light to “diffuse” through several centimeters of tissue. Above 900 nm, water absorption is significant and light delivery can be heavily attenuated. Thus, the limited penetration depth of light for currently available opsin (excitation 400-600 nm) is a major concern, especially when considering the high visible-light absorption (400-600) of hemoglobin/blood cells through the blood-brain bather (BBB), neurovasculature and even chronic BBB leakage if the optic waveguides are implanted into the brain.

Optogenetics. Optogenetics relies on the insertion of light-sensitive ion channels to activate neurons. One of the strengths of optogenetics is that it offers a genetic selectivity of cell types. However, this method has several major limitations, including, but not limited to: 1) requiring genetic manipulation; 2) limited depth penetration of light (450-560 for excitation opsin channel variants) into highly scattering biological tissue; and 3) poor spatial selectivity of genetically similar, but functionally different neurons (e.g., two excitatory neurons with different directional tuning properties) caused by highly scattering biological tissue and/or poor control over virus diffusion radius and virus transduction rate. This prevents activating the same discrete group of neurons over time.

Infrared (IR) Stimulation. IR neural stimulation in the brain provides a number of challenges due to poor mechanistic understanding and dramatic anatomical differences between brain and peripheral nerves. Further, it has limited penetration depth (300-600 μm) due to high laser power requirements and stimulation frequency (<4 Hz) due to tissue heating induced injury. Laser power requirements can be reduced by using micro-waveguides and materials with high photovoltaic properties.

Radio-Frequency and Ultrasonic Stimulation. While alternatives exist using “antennas” to convert RF or ultrasonic energy into electrical current, their chronic biocompatibility in vivo require further evaluation and development. Further, these stimulation modes are unable to selectively stimulate individual channels in multi-implant “arrays” since near-field RF excitation is difficult to focus below several centimeters resolution.

Need. For many basic neuroscience studies, including learning and plasticity, it is desirable to precisely interrogate the same discrete populations of neurons longitudinally. For example, to probe how specific neuronal circuits change after learning and training paradigms, it is often necessary to be able to stimulate the same neurons over time and map out changes in the network activity.

Impact. Developing advanced neural probes for long-term (e.g., permanent), spatially selective, efficient, and reliable neural stimulation could potentially lead to paradigm shifts in both neuroscience research and clinical neurotechnologies. The capability to activate a selective brain region for long periods of time with great precision would be a powerful tool in neuroscience research for linking low-level neuronal circuits to high-level brain network function, such as learning, memory, and perception.

As such, disclosed herein are untethered/free-floating photovoltaic and/or photothermal microelectrode for long-term, stable, chronic electrical stimulation of the same discrete populations of neurons over weeks and months is described. A disclosed electrode can be ultra-small (such as 0.001 to 25 microns) and mechanically uncoupled from the skull, e.g., implanted directly in neuronal tissue. The disclosed devices can be uninsulated, partially insulated, or fully insulated with a soft thin dielectric layer, which can be covalently functionalized with neuron-binding bioactive coatings and nanostructures. The dielectric can further incorporate optical filters, such as those employed in fluorescent microscopy for large scale arrays with selective wavelength stimulation capabilities. The disclosed electrodes can be made of electrochemically stable material or designed to optimize non-faradaic charge transfer. An additional layer of spatial selectivity can be achieved using a piezomotor driven optical waveguided to focus light onto the electrode.

The disclosed electrodes and methods have several advantages compared to traditional electrical stimulation. One advantage is that the photovoltaic cell electrodes are mechanically decoupled from the skull. This reduces modes of mechanical failure, inflammation, and electrode position movement due to micromotion between the brain and the skull. This leads to reduced glial scarring, more stable neural interfaces, and better chronic charge transfer. Furthermore, the free floating electrodes can be anchored to their nearby neurons through neuron binding nanostructures or bioactive coatings. In some embodiments, sub-cellular size photovoltaic nanoparticle electrodes of appropriate concentration (to avoid cytotoxicity) are utilized that can be engulfed by neurons for intracellular neural activation. In addition, photovoltaic cell electrodes (including those in a photodiode configuration) can be spherically designed to have even charge density distribution across the entire electrode and a more localized field of activation in the tissue. This enables stimulation of the same discrete population of neurons over time, which is a powerful new tool for probing neural circuits in memory, learning, training, and plasticity studies. Some additionally advantageous features of the disclosed electrodes are the following: i) ultra-small (sub-cellular), ii) can have sophisticated surfaces tailored for specific biological processes and to anchor to neurons, iii) uncouple the mechanical attributes necessary to insert the probe from those that are best for long-term implants, iv) can be optically selective for specific wavelengths and compatible with existing optogenetic techniques.

Moreover, using a cell-sized or sub-cellular-sized photovoltaic and/or photothermal electrodes activated by IR light drastically reduces the power requirements for neural activation, improves light penetration depth from longer wavelengths, and dramatically improves X-, Y-, and Z-axis spatial resolution. Because the photovoltaic and/or photothermal stimulating electrodes use IR wavelengths and have small optical shadow footprints, this technology can also be used with optogenetic technology to observe broad activation, then probe the activation or silencing of specific cell types longitudinally.

It is contemplated that the disclosed electrodes and methods of use will directly impact the basic neuroscience research community that uses chronic neural microelectrode technologies (neuroscientists and neural engineers), as well as clinical science communities that use neural interface devices and engineers and scientists that are developing biomaterials and devices for interfacing with the brain, spinal cord, and peripheral nerves. In addition, the disclosed electrodes and methods enhance basic neuroscience communities that use optogenetics by providing tools to probe spatial selectivity of neural activation to the genetic selectivity of opsin constructs with non-overlapping wavelength sensitivity. The disclosed electrodes and methods of use are also expected to impact controlled drug delivery and in particular, cancer therapeutics.

II. Electrodes

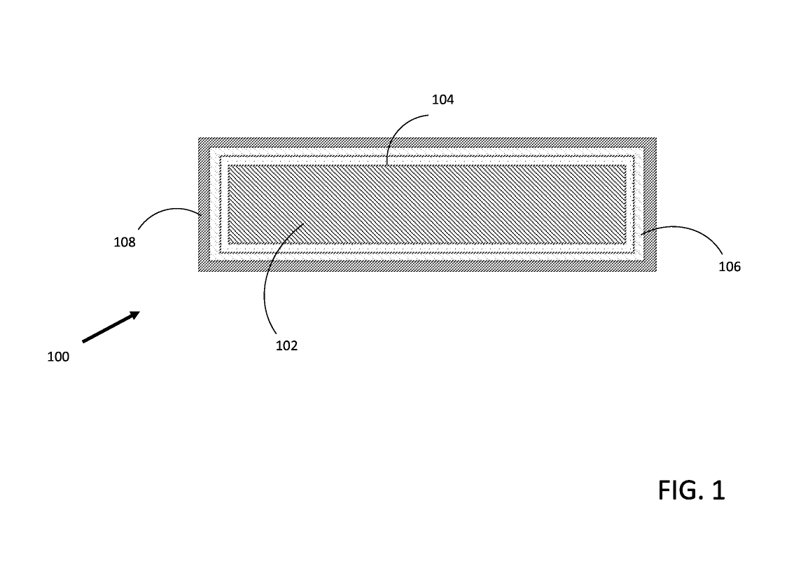

With reference to FIG. 1, a cross-sectional view of an exemplary untethered/free-floating photovoltaic microelectrode 100 is provided. FIG. 1 illustrates an electrode body 102, an optical filter coating 104, a biocompatible dielectric coating 106, and a bioactive coating 108. In some embodiments, electrode 100 has at least one critical dimension, such as diameter, diagonal, width, thickness, height, length, or lattice diameter, smaller than a target cell, such as a neuronal cell (an electrically excitable cell of the nervous system, typical size of 15-30 microns in diameter). For example, the diameter or diagonal of the electrode 2 can be between 0.001 and 25 microns, such as between 0.01 and 20 microns, 1 and 15 microns, 5 and 10 microns, including 0.001 microns, 0.01 microns, 1 micron, 2 microns, 3 microns, 4 microns, 5 microns, 6 microns, 7 microns, 8 microns, 9 microns, 10 microns, 11 microns, 12 microns, 13 microns, 14 microns, 15 microns, 16 microns, 17 microns, 18 microns, 19 microns, 20 microns, 21 microns, 22 microns, 23 microns, 24 microns or 25 microns. The length or width of electrode 100 can likewise have a subcellular dimension. Electrode 100 can comprise any suitable shape, such as a rod, rectangular prism, pyramid, or sphere.

Electrode body 102 can be made from a photoelectric and/or photothermal material to stimulate neurons with multiple fully implantable untethered free-floating cellular or sub-cellular size photovoltaic converters with neuron binding and anchoring nanostructured coatings that can be optically tuned to selectively activate at specific wavelength bands.

The photoelectric effect is a phenomenon of conductor or semiconductor materials. Light energy excites an electron in the valence band to the conduction band, and generates an electron-hole pair resulting in an electric voltage. In solar cells, electron transfer occurs through electrons in the conduction band flowing to an electron acceptor. Then, other electrons combine with the holes in the semiconductor to generate a current flow. This electrical current or voltage generated from a wireless light source can, in turn, electrically stimulate nearby neurons to elicit a physiological response. In photothermal excitation, some or all of the energy from the photon and electron collision is released as heat.

The photoelectric effect is the creation of voltage or current in a material upon exposure to light energy first observed by Heinrich Hertz in 1887, and described by Albert Einstein in 1905. One characteristic of the photoelectric effect is that electrons are only dislodged by the photoelectric effect if light reaches or exceeds a threshold frequency, below which no electrons can be emitted from the electric conductor regardless of the amplitude and temporal length of exposure of light. This can be described by the equation(s):

E=hv=hc/λ [1]

ψ=hv₀=hc/λ₀ [2]

where E is the energy of the absorbed photon, v is frequency of the light, h is Plank's constant, c is the speed of light, λ is the wavelength of the light, and ψ is the photoelectric work function (see Table 1).

TABLE 1
Single-Photon Photoelectric Work Functions

| Material | Work Function (eV) | Wavelength (nm) |
|---|---|---|
| Carbon | 4.81-5.2# | 257-238# |
| Gold | 5.1 | 243 |
| Platinum | 6.35 | 195 |
| Silicon | * | * |

* Depends on dopants.
#Range can increase by doping

Additionally, v₀ and λ₀ are the light frequency and wavelength threshold necessary to generate photoelectricity, respectively. The photoelectric effect is observed when E>ψ for single photon events. Further, the generated current is proportional to:

j=hvΦ [3]

where j is current and Φ is photon flux, which is proportional to intensity/area. However, these cutoff frequency and wavelengths described by Einstein only apply to single-photon events. Maria Goppert-Mayer described that multi-photon events, where two or more photons simultaneously collide at the target, can greatly reduce the cutoff threshold. In multi-photon photoelectric activation, E does not scale linearly with N. Instead E_{N} can be described as:

E_{N}=(N+S)hv [4]

where N is the number of simultaneously colliding photons and S is some positive scalar value. This was further described and characterized as:

E_{N}=(Nhv)/(1−Bᵥf(I)) [5]

where f(I) is a function of light intensity and Bᵥ is a coefficient. This experimentally validated expression represents the quantum level photon-photon interaction for high intensity light. Essentially, even in a single photon beam, two single-photons can “entangle” to from a single biphoton ‘packet’. The unexpected advantage is that for N-photon events, longer wavelengths can be used to generate the photoelectric effect than would be predicted by multiplying the cutoff wavelength by N. Further, laser power injection into the tissue decreases with increases in wavelength. This means that more photoelectric events can be generated with longer wavelength while remaining within tissue heating desired limits. Thus, photovoltaic stimulation is advantageous for at least the following reasons: 1) high spatial selectivity in terms of stimulated neuronal density, 2) high reliability in terms of consistently stimulating a targeted number of neurons over the entirety of the study period, and 3) excellent biocompatibility by reducing glial scarring and maintaining high-quality charge transfer properties. Henceforth, photoelectric excitation is implied to be achieved by any optical means such as single-, two-, bi-, multi-photon methods that produce a current or voltage in the material.

In some embodiments, electrode body 102 is formed of a material with at least high photovoltaic properties (pseudocapacitive or capacitive charge transfer) and/or photothermal (efficient thermal conversion). In some examples, the photovoltaic and/or photoelectric material exhibits low photogalvanic and photoelectrochemical properties (Faradaic charge transfer) that can cause damage to the material and tissue. In some examples, electrode body 102 does not degrade or degrades minimally over time (such as weeks or years) due to the high photovoltaic and/or photothermal properties.

Photogalvanic activation occurs when an electron is separated from the hole associated with it leading to the generation of current. Photoelectrochemical activation occurs when the separation of an electron from a hole induces an oxidation reaction. Photovoltaic activation occurs when an electron's energy level is increased by the collision with photon(s), leading to an increase in voltage, but the electron is not separated from its hole. This means that non-Faradaic charge transfer occurs, because the electron remains associated with its original atom or hole. During non-Faradaic charge transfer the electron returns to its original hole. Non-Faradaic charge transfer is preferred to minimize electrode and tissue damage caused by electrons or ions participating in unwanted side reactions. These losses can appear as heat, chemical byproducts and/or loss of electrode. Furthermore, with non-Faradaic charge transfer a balancing pulse is not necessary to maintain charge balance, as the generation of voltage is charge neutral. In some embodiments, electrode body 102 is configured to produce 0-1V when illuminated by laser light, such as 0.1V, 0.2V, 0.3V, 0.4V, 0.5V, 0.6V, 0.7V, 0.8V, 0.9V, or 1.0V.

In some embodiments, it is desirable for the electrode to degrade over time. If it degrades too quickly, however, it could damage the tissue or prematurely end the treatment. Accordingly, the amount of photogalvanic or photoelectrochemical response of the electrode depends on the circumstances of the use. A slow degradation of the electrode can be desirable such that the electrode dissolves and need not be removed. The speed of degradation depends on parameters, such as the target tissue, size of electrode, length of therapy, number of stimulations, among other possible parameters.

In some embodiments, electrode body 102 is formed of a material with photothermal properties. The photothermal effect in general is produced by the photoexcitation of material, resulting in the production of thermal energy (heat). For example, heating of a disclosed electrode generates a thermal gradient which in turn forces ion channels to open and cause depolarization. For example, heating of a disclosed electrode generates a thermal gradient which in turn forces ion channels to open and cause depolarization. The photothermal converters are an efficient energy converter and photon scavenger that focuses energy conversion at the electrode site that would normally be scattered by blood vessels. In some examples, electrode body 102 is formed from metallic nanoparticles, such as gold nanoparticles. The gold nanoparticles can be densely packed into nanostructures using layer-by-layer composite assembly (LbL).

In some embodiments, electrode body 102 is formed from carbon nanotube (CNT), carbon fiber, graphene, doped diamond, or a combination thereof. Carbon nanotubes can be single-walled (SWNT), double-walled (DWNT), or multi-walled (MWNT). Carbon fibers can be made from CNT bundles. CNT and carbon fibers possess a wide range of direct bandgaps depending on their chiral vector (m,n) orientations. For example, for (n, m), CNTs are metallic (n=m), quasi-metallic (n−m=3X, where X is an integer), and semiconducting (others). CNTs possess strong photoabsorption from IR to ultraviolet wavelengths, high power conversion efficiency, very high non-Faradaic charge transfer, very low Faradaic charge transfer, high carrier mobility, and reduced carrier transport scattering. Specifically, carbon has nearly an order of magnitude higher capacitive charge transfer capacity, as well as over 8 orders of magnitude greater resistance than traditional metals, indicating minimized Faradaic charge transfer—which could otherwise damage the tissue or electrode material. This makes carbon a desirable photovoltaic material as well as a great electrical stimulation electrode material. Furthermore, carbon and CNTs also have tunable photovoltaic properties through the incorporation of dopants. SWNTs can form ideal p-n junction photovoltaic diodes that reach the theoretical limits. Under illumination, SWNT diodes show significant power conversion efficiencies owing to enhanced properties of an ideal diode. The photoelectric effect involves light energy exciting an electron in the valence band to the conduction band, and generating an electron-hole pair resulting in an electric voltage. The carbon fiber, or CNT, serves as a photovoltaic converter that converts photon to electrical voltage, which in turn electrically stimulates nearby neurons. This mode of neuronal excitation is very efficient requiring relatively low power, has greater penetration depth, and reduces risk of thermal injury compared to IR neural stimulation.

In some embodiments, electrode body 102 is formed from a doped composition, such as doped silicon or doped CNT. Silicon can be p-doped, to remove electrons, for example using boron, or n-doped, for example using phosphorous, to add electrons. Doped silicon can be produced by, for example, chemical vapor deposition processes to yield an amorphous, polycrystalline, or nanocrystalline structure. In some examples, when a negative voltage is applied to p-type silicon, major carriers become depleted from the surface. Upon illumination with a laser, photogenerated currents are driven to the surface. Laser intensity, pulse duration, and pulse frequency can be tuned to control the generated photocurrent. In some examples, electrode body 102 is made from multiple layers of doped silicon forming p-n junctions or p-i-n junctions. These junctions can form photo-diodes where upon illumination with a laser, photogenerated currents are driven to the surface. Laser intensity, pulse duration, and pulse frequency can be tuned to control the generated photocurrent.

In some embodiments, electrode body 102 is made from a combination of CNT and doped silicon. For example, coating n-doped silicon with SWNTs can enhance power conversion efficiency. In some embodiments, electrode body 102 is made from other biocompatible materials, such as gold, platinum, iridium, or platinum/iridium alloy. In some embodiments, electrode body 102 is mounted onto Indium Tin Oxide (ITO), Indium Trioxide (In2O3) or 1-5 layer thick graphene sheet coated glass slides. ITO, In2O3, and thin graphene are electrical conductors with very high phototransparency (ITO in visible range; In2O3 in the IR range), so the photoelectric or photothermal excitation rate is very low (photons pass through the material at a greater rate instead of all photons colliding with electrons in the material).

In some embodiments, electrode body 102 of electrode 100 is coated with an optical coating 104. For examples, optical coating 104 can be applied to electrode body 102 to allow only certain wavelengths of light to penetrate causing the photovoltaic activation of electrode 100, i.e. filter out unwanted wavelengths of light. For example, optical coating 104 can be Red 40, Blue 1, Solvent Red 8 and/or Solvent Blue 36. It is contemplated that other coatings can be used to allow selective transmission of wavelengths of light. In some examples, optical coating 104 is tuned to allow transmission of wavelengths of light with wavelengths between 400 and 2000 nanometers, such as between 550 and 900 nanometers. In other examples, the allowed wavelength of light is the center wavelength (CWL). The CWL can be any discrete wavelength between 400 and 2000 nanometers. Optical coating 104 allows light at the CWL to pass and light within the passband, i.e. bandwidth, to also pass. The bandwidth of transmitted light can be between 1 and 100 nanometers, such as 1 nm, 5 nm, 10 nm, 20 nm, 30 nm, 30 nm, 40 nm, 50 nm, 60 nm, 70 nm, 80 nm, 90 nm, 100 nm. For example, an optical coating with a CWL of 900 nanometers and bandwidth of 50 nanometers can allow light between 850 and 950 nanometers to pass. A narrow bandwidth allows selective activation of electrodes using different wavelengths of light that are in close proximity to each other.

In some embodiments, electrode body 102 of electrode 100 is coated with a dielectric coating 106 and/or thermal insulator coating, such as by using chemical vapor deposition. In some examples, dielectric coating 106 is one or more biocompatible coatings, such as parylene, polyimide, silicon oxide, or polydimethylsiloxane (PDMS). It is contemplated that other biocompatible dielectric coating can be used to form a capacitive layer between electrode body 102 and tissue. In some examples, dielectric coating 106 insulates electrode body 102 to enable capacitive charge transfer between the electrode and surrounding tissue. It can also protect the electrode from inflammation responses and provide an added level of biocompatibility. In some examples, dielectric coating 106 is between 10 nm and 10 microns thick, such as between 10 nm to 1 micron, 100 nm to 10 microns, or 100 nm to 1 micron, including 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 micron, 2 microns, 3 microns, 4 microns, 5 microns, 6 microns, 7 microns, 8 microns, 9 microns and 10 microns.

In some embodiments, electrode body 102 of electrode 100 is coated with a bioactive coating 108 to maintain close spatial proximity between electrode 100 and a target cell. For example, bioactive coating 108 can be neuron binding proteins, such as L1 cell adhesion molecule (L1CAM), laminin, collagen I, collagen IV, fibrinogen, n-cadherin, and/or neural cell adhesion molecule (NCAM). Close spatial proximity between electrode 100 and a target cell can reduce the required stimulation amplitude, ensuring electrical stimulation is within desired limits, and increase spatial selectivity. In some embodiments, the bioactive coating is a nanostructured coating with surface textures to maintain close spatial proximity between electrode 100 and a target cell, and to improve charge injection capacity. Bioactive and nanostructured coatings applied to electrodes are advantageous for at least the following reasons: 1) anchor the probe to the target tissue; 2) improve charge transfer to neurons by reducing the distance to nearby neurons; and 3) attenuate targeted reactive tissue response. These advantages improve the efficacy, stability, and biocompatibility of the stimulation electrode.

In some embodiments, optical coating 104 and dielectric coating 106 are integrated together to form a single coating. In other embodiments, bioactive coating 108 is integrated with optical coating 104 and/or dielectric coating 106. In still further embodiments, optical coating 104, dielectric coating 106 and bioactive coating 108 are combined to form a single coating. Furthermore, any combination of coatings 104, 106, and 108 can be applied to electrode body 102. For example, only a single coating can be applied to electrode body 102. In some embodiments, one or more coatings, or combinations of coatings, can be applied to a portion of electrode body 102. For example, bioactive coating 108 can be applied only to a surface of electrode body 102 that is to be in closest contact with the target cell.

The disclosed electrodes are designed so that they are free floating (untethered/non-wired) when in use, such as in tissue. Untethered, non-wired electrodes are advantageous for at least the following reasons: 1) reduce mechanical failure modes including lead breakage; 2) reduce movement of the electrode with respect to the bulk tissue; 3) reduce chronic inflammation and glia scarring via decoupling the tether by the electrical lead; and 4) allow for better chronic spatial selectivity of excitable cells through microscale photovoltaic and/or photothermal electrodes without shunt leakage. These combined advantages improve functional longevity, biocompatibility, reliability, and stability of the electrode.

Dopants and optical coatings on photovoltaic and/or photothermal electrodes provide sensitivity to selective wavelengths, which are advantageous for at least the following reasons: 1) provide for differential modulation through the use of multiple wavelength channels; 2) provide for combination studies (e.g., optogenetics), and 3) materials can be tuned for preferential non-Faradaic charge transfer. Combined, these advantages improve the stimulation energy transfer efficiency, material stability/longevity, biocompatibility, and utility (e.g. multi-channel).

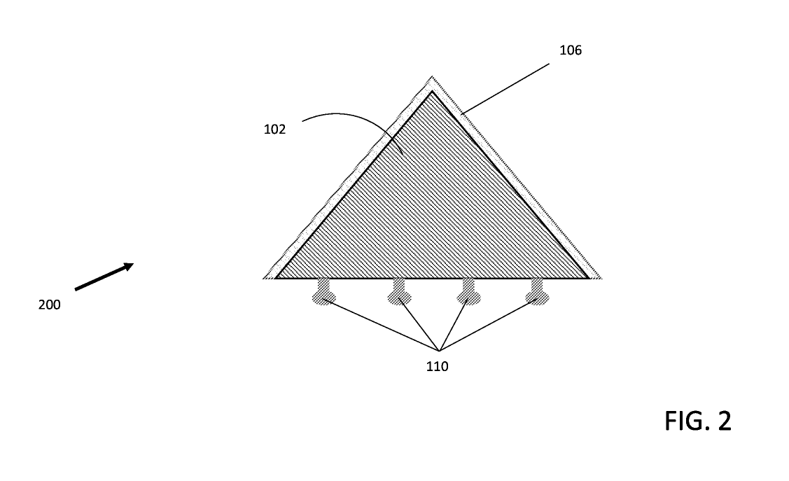

FIGS. 2-4 show example embodiments of electrode 100 with varying shapes, coatings and surface textures. With reference to FIG. 2, a cross-sectional view of an exemplary untethered/free-floating photovoltaic microelectrode 200 is provided. FIG. 2 illustrates electrode body 102, dielectric coating 106, and surface textures 110. In some embodiments, electrode body 102 has multiple light catching surfaces at different angles from each other. In some examples, electrode body 102 is pyramidal shaped, such as a polyhedron, a tetrahedron, star pyramid, triangular prism, or other suitable multisurfaced shape.

In some embodiments, electrode 102 of electrode 200 is coated with a combined coating covering some or all of electrode body 102. In some examples, the combined coating comprises a biocompatible dielectric 106, a bioactive coating 108, and an optical coating 104, or any combination thereof. In some examples, the coating covers fewer than all the surfaces of electrode body 102, such as only the surfaces that are not in closest proximity to the target cell. In some examples, the coating covers some or all surfaces of electrode 102, but can have regions where the coating is thinner.

In some embodiments, electrode body 102 of electrode 200 has surface textures 110. In some embodiments, surface textures 110 are smaller than 1 micron, such as 500 nm, in all dimensions that are to be engulfed into a target cell. In some examples, surface textures 110 are mushroom shaped nanostructure protrusions on the surface of the electrode which tightly trap neurons as they engulf the protrusion through endocytosis, e.g. mechanisms similar to clathrin mediated endocytosis or phagocytosis. In other examples, surface textures can be ridges, spike, nails, or other suitable shape. Nanostructures can be gold mushroom-shaped microelectrodes (gMμE); functionalized gold-spine electrodes; CNTs; carbon fibers; Titanium (IV) nitride (Ti3N4) or tungsten micro-nail electrodes; doped silicon; boron nitride nanotubes (BNNT); or other suitable material that can be engulfed by the target cell, such as extracellular matrix proteins, e g laminin and collagen IV.

With reference to FIG. 3, a cross-sectional view of an exemplary untethered/free-floating photovoltaic microelectrode 300 is provided. FIG. 3 illustrates an electrode body 102 and coating. In some embodiments electrode 102 has a circular, or elliptical, cross-sectional area. In some examples, electrode body 102 is a rod, cone, sphere, or other curved surface shape. In some embodiments, electrode 300 has a coating 108 functionalized with bioactive material. In some examples, coating 108 can be combined with a dielectric coating, an optical filter coating, or both. Bioactive material is functionalized with coating 108 to promote target cell adhesion. The bioactive material can be binding proteins such as L1 Cell Adhesion Molecule (L1CAM), neural cell adhesion molecule (NCAM), Ig (immunoglobulin) superfamily (IgSF CAMs), integrins, cadherins, and/or selectins. In some examples, coating 108 covers all surfaces of electrode body 102. In other examples, coating 108 covers only a selected portion of electrode body 102.

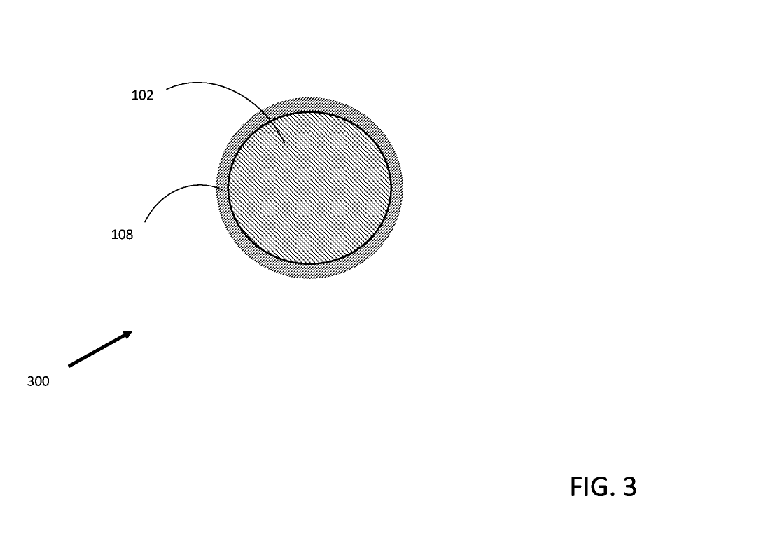

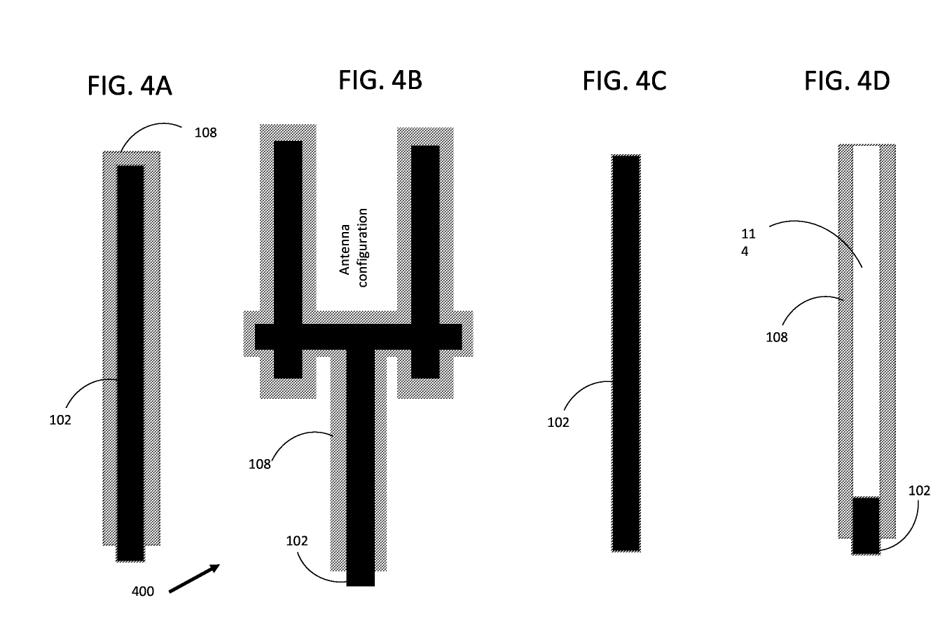

With reference to FIGS. 4A-4D, several cross-sectional views of embodiments of electrode 400. In some embodiments, electrode 102 has a rectangular cross-section (as shown). In other embodiments, electrode 102 has shapes as described above. FIG. 4A illustrates an embodiment of electrode 400 partially coated by coating 410. In some examples, electrode body 102 is exposed, i.e. not coated, at end 402. Exposing a portion of electrode 102 can increase the capacitive coupling at that point between the target cell and electrode 400.

FIG. 4B illustrates an embodiment of electrode 400 with an antenna array. The extended insulated region allows for increase photon scavenging of scattered light as it passes through the tissue (tissue/water scatters light). The electrical or thermal energy is then transferred to the tissue at the electrode 102.

FIG. 4C illustrates an embodiment of electrode 400 is not coated. In some embodiments, the photovoltaic and/or photothermal material is activated by a specific wavelength of light. In other embodiments electrode 400 has surface textures to promote cell adhesion. In still other embodiments, electrode 400 is a carbon fiber. In some examples, electrode 400 can stimulate target cells along its entire length.

FIG. 4D illustrates an embodiment of electrode 400 with an integrated micro-waveguide 114. In some embodiments, micro-waveguide 114 is a single-mode or multi-mode fiber that can be split into multiple waveguides and converge multiple wavelength lasers. Micro-waveguide 114 can enable complex combination or selective wavelength activation of adjacent photovoltaic electrodes, such as electrode body 102. The path of laser light can be modified to enhance entanglement of photons, e.g. biphotons, using micro-waveguide 114. Biphotons delivered through a single-mode and or multi-mode fiber further increase efficiency compared to the same intensity of coherent light in two-photon processes. Micro-waveguide 114 increases the spatial selectivity when activating electrode body 102 with laser light by causing the electrode to adhere to the target cell. In some embodiments, micro-waveguide 114 is coated with coating 108. It is contemplated that any of the electrodes shown in FIGS. 4A, 4B and 4D may include additional coatings as described above, such as combined coatings with a dielectric coating, an optical filter coating, or both. It is also contemplated that electrodes with configurations shown in FIGS. 4A, 4B or 4D may include a dielectric coating, an optical filter coating, or both without a biocompatible coating.

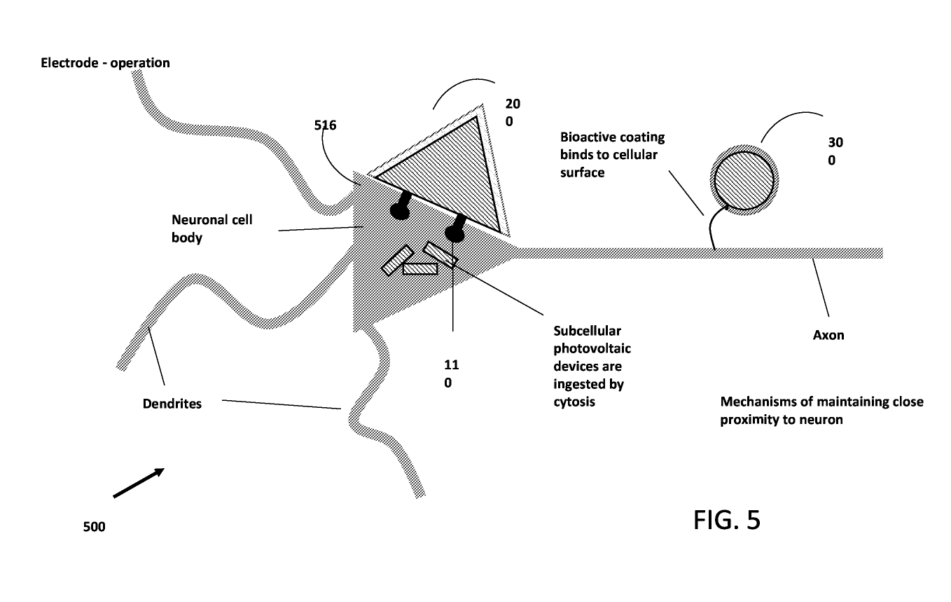

With reference to FIG. 5, various embodiments of untethered/wireless electrodes are shown in close proximity to a target cell. In some examples, the target cell is a neuron in the brain. In other examples, the target cell is an electrically excitable cell of the nervous system, which includes the brain, cranial or peripheral nerves, spinal cord, and the ganglia of the peripheral nervous system (PNS), which comprise the central nervous system (CNS). FIG. 5 shows neuron 516 comprising a cell body, an axon, and dendrites. In some embodiments, electrode 200 is in close proximity to the cell body. Electrode 200 can have surface textures that promote endocytosis. In some examples, the surface textures are mushroom shaped protrusions 110 as described above. The protrusions can be engulfed by the cell such that they extend across the cell membrane with a distal end being inside the cell and a proximal end being connected to the electrode body. In other examples, the surface textures can be ridges, nanotextures, or other shapes that promote cell adhesion. In some examples, electrode 200 is coated by a coating on the surfaces not in close proximity to the target cell. Not coating the surface in proximity to the cell can focus charge/energy transfer towards a preferential direction. In other examples, the entire electrode 200 is coated with a coating.

In some embodiments, electrode 300 is covered with a bioactive coating. In some examples the coating is L1 CAM and promotes cell adhesion. In some examples, the coating is combined with a dielectric coating and/or an optic filter coating. The bioactive coating promotes cell adhesion and maintains electrode 300 in close proximity to the cell. In some examples the bioactive coating promotes adhesion to the axon, and in other examples it can promote adhesion to the cell body.

In some embodiments, untethered/wireless photovoltaic and/or photothermal electrodes are small enough to be completely engulfed by the cell by endocytosis or phagocytosis. In some examples, receptor-mediated endocytosis occurs when nanoparticles, such as particles smaller than 1 micron, such as 500 nm, contact the cell membrane. Once the particles are engulfed, cells can be stimulated intracellularly. In some examples, exemplary electrodes smaller than 1 micron, such as smaller than 500 nm, in all dimensions allow the electrode to be engulfed by the cell. In some examples, such electrode is coated with at least one of a dielectric coating, an optical filter coating, and/or a bioactive coating. With a disclosed electrode inside cell 516, it can be activated with light to stimulate the cell.

The disclosed photovoltaic and/or photothermal electrodes produce a voltage that directly stimulates excitable tissue. This mode of wireless stimulation is advantageous for at least the following reasons: 1) the use of far-red to IR spectra allows for greater depth penetration; 2) the use of photovoltaic and/or photothermal converters allow for deeper depth penetration than IR neural stimulation; 3) photovoltaic converters drastically reduce power consumption and tissue heating, enabling much higher stimulation frequencies; 4) excitation density can be modulated by light power; 5) risks associated with genetic manipulation are avoided; and 6) the use of far-red to IR light enables compatibility and combination studies with other stimulation modes such as optogenetics. Combined, these advantages improve stimulation efficacy, energy transfer efficiency, and utility (e.g., depth penetration).

III. Methods of Fabricating Electrodes

Disclosed are methods of making photovoltaic and/or photothermal microelectrode for stimulating neuronal tissue, or other excitable tissue. In some embodiments, the method includes fabricating carbon fibers and/or CNT. (see, for example, Kozai, T. D. Y, and Vazquez, A. L, “Photoelectric artefact from opotgenetics and imaging on microelectrodes and bioelectronics: new challenges and opportunities,” J. Materials Chemistry B., 2015, 3: 4965-4978; Seymour J P, Kipke D R, “Neural probe design for reduced tissue encapsulation in CNS,” Biomaterials, 2007 September; 28(25):3594-607. Epub 2007 Apr. 5, which is hereby incorporated by reference in its entirety; see also Nanotechnology and Neuroscience: Nano-electronic, Photonic and Mechanical Neuronal Interfacing, Chapter: Nanostructured Coatings for Improved Charge Delivery to Neurons, Publisher: Springer Science+Business Media New York, Editors: Massimo De Vittorio, Luigi Martiradonna, John Assad, pp. 71-134, each of which is hereby incorporated by reference in its entirety; see also U.S. patent application Ser. No. 13/805,132, which is hereby incorporated by reference in its entirety). In some examples, carbon is fabricated in tubular or fiber structures; therefore its parameter space is restrained to diameter and length. In some examples, carbon fibers, with different dopants and (n,m) indices, between 5 and 10 microns in diameter are utilized. In some examples, fibers are cut into their target lengths using a suitable implement or method, such as a cryostat, razor, scissors, electron beam, plasma etching, or burned with fire. In additional examples, graphene sheets of varying size and thickness from sonication, SWNT, DWNT, MWNT of varying (n,m) orientations, diameter, lengths, and dopants are utilized.

In some embodiments, the method includes fabricating doped silicon photodiodes, including fabricating layers of n-doped and p-doped silicon. (see U.S. Pat. No. 5,397,350, which is hereby incorporated by reference in its entirety; see also U.S. Pat. No. 5,556,423, which is hereby incorporated by reference in its entirety). Silicon has a greater design space and can be fabricated into various shapes. Implantable silicon “fibers” with the same dimensions as those of carbon are contemplated, as well as solid silicon spheres spanning a diameter between 5 microns and 30 microns in diameter. Sub-cellular, cellular, and high aspect geometry photovoltaic and/or photothermal electrodes can be etched from n-type or p-type photosensitive silicon wafers using traditional lithographic fabrication methods. Moderately doped silicon, such as between 0.01 Ohm-cm and 1 Ohm-cm, recording arrays form excellent photoconverters. By contrast, degeneratively doped silicon, such as about 0.001 Ohm-cm, is immune to even large pulses of laser light. Microstimulation amplitude can be a function of doping and substrate geometry. Silicon-on-insulator (SOI) wafers having doping values of 0.01, 0.1, and 1 Ohm-cm using standard “Michigan” process is contemplated. (see Wu F. et al., “An implantable neural probe with monolithically integrated dielectric waveguide and recording electrodes for optogenetics applications,” J Neural Eng. 2013 October; 10(5):056012. doi: 10.1088/1741-2560/10/5/056012. Epub 2013 Aug. 28, which is hereby incorporated by reference in its entirety). In some examples, patterned devices are released from the wafer using a standard DRIE etch process.

Disclosed are methods of fabricating micro-waveguides for focusing laser light, splitting laser light, and combining laser light. In some embodiments, micro-waveguides are fabricated and connected to a fiber optic cable receiving laser light from a laser. In another embodiment, micro-waveguides are connected to photovoltaic and/or photothermal microelectrodes to focus light directly onto the electrode body. For exemplary fabrication methods see Wu F. et al. (“An implantable neural probe with monolithically integrated dielectric waveguide and recording electrodes for optogenetics applications,” J Neural Eng. 2013 October; 10(5):056012. doi: 10.1088/1741-2560/10/5/056012. Epub 2013 Aug. 28, which is hereby incorporated by reference in its entirety).

Disclosed are methods of coating photovoltaic microelectrodes comprised of CNT, carbon fiber, graphene, doped silicon, and/or doped CNT. In some embodiments, a dielectric coating is applied to the microelectrode or to another coating. In some examples, an optical coating is applied to the microelectrode or to another coating. In some examples, a bioactive coating is applied to the microelectrode or to another coating. In some examples, the combined thickness of the one or more coatings is between 0 micron and 25 microns, such as between 0.8 microns and 5 microns, including 0.1, 0.3, 0.5, 0.8, 1, 2, 5, 10, 20 and 25 microns, to allow for adequate capacitive coupling of charge form the microelectrode. Methods of applying any of the aforementioned coatings are found in Kozai T D, et al. (“Ultrasmall implantable composite microelectrodes with bioactive surfaces for chronic neural interfaces,” Nat Mater. 2012 December; 11(12):1065-73. doi: 10.1038/nmat3468. Epub 2012 Nov. 11, which is hereby incorporated by reference in its entirety).

Disclosed are methods of fabricating surface textures on photovoltaic microelectrodes comprised of CNT, carbon fiber, graphene, doped silicon, and/or doped CNT. In some embodiments, surface textures, such as mushroom-shaped protrusions, are fabricated onto the microelectrode body and are smaller than 1 micron. In some embodiments, surface textures are ridges, spike, and /or nails. Surface textures can be fabricated onto the photovoltaic microelectrode with processes found in Nanotechnology and Neuroscience: Nano-electronic, Photonic and Mechanical Neuronal Interfacing (Chapter: Nanostructured Coatings for Improved Charge Delivery to Neurons, Publisher: Springer Science+Business Media New York, Editors: Massimo De Vittorio, Luigi Martiradonna, John Assad, pp. 71-134, which is hereby incorporated by reference in its entirety).

IV. Methods of Using Disclosed Electrode

Methods of using the disclosed electrodes are provided. In some embodiments, laser light is used to activate a disclosed untethered/free-floating photovoltaic and/or photothermal microelectrodes. In some examples, an ultrafast coherent laser is used to produce the laser light. Ultrafast coherent lasers produce laser light with repetition rates from 10 Hz to 100 MHz and pulse energies from nJ-level to 100's mJ. Ultrafast coherent lasers also generate very short pulse widths—on the order of nanoseconds to femtoseconds. As pulse width decreases, the stimulation can become more efficient. This further enables eliciting physiological effects using very short pulse width and high voltage stimulation paradigms, while minimizing the kinetics of irreversible Faradaic charge transfer.

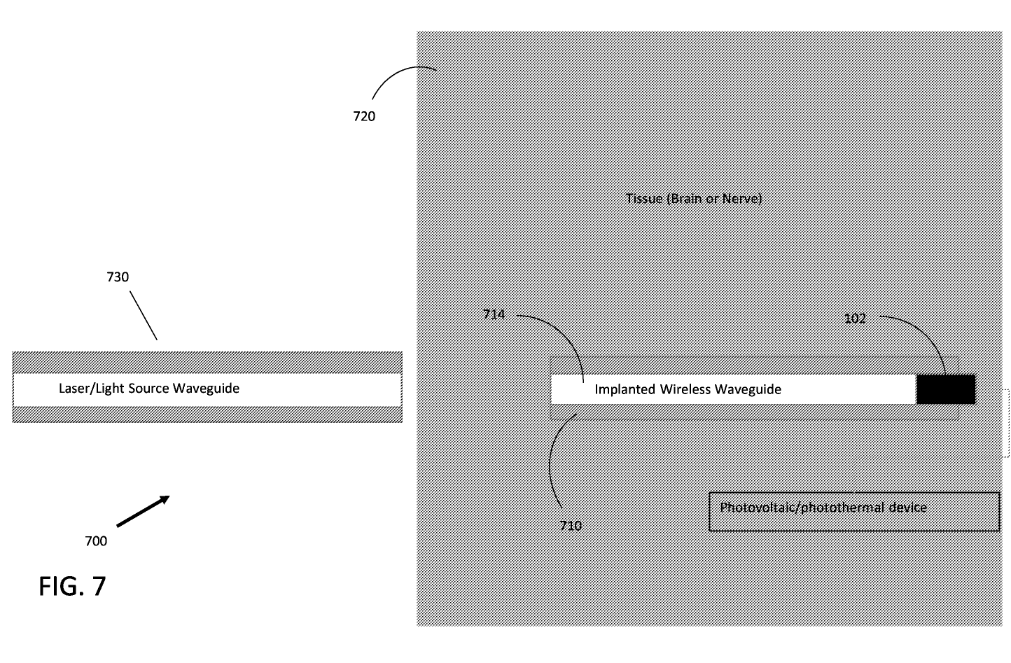

FIGS. 7-9 show various methods of stimulating target cells with exemplary untethered/wireless photovoltaic and/or photothermal microelectrodes. With reference to FIG. 7, an exemplary microelectrode 102 is implanted in tissue 720 and stimulated by a laser or other light source 730 is shown. In some embodiments, electrode 102 is implanted into neuronal tissue, such as brain or central nervous tissue. Specific insertion tools and techniques that are required for reliable insertion of the photovoltaic electrodes into the brain of rodents are contemplated. The functional specifications for insertion include, but are not limited to, spatial precision (depth, positioning) matching that of conventional sharp wire microelectrodes, which is nominally less than 10 microns and compatibility with existing micromanipulators and typical surgical techniques. Insertion of electrodes can be first tested in 0.5% agarose. In some embodiments, a 5-10 micron tip glass pipette is used to inject the photovoltaic electrodes using a pressure injector. In some embodiments, a dissolvable microelectrode insertion shuttle is used for inserting ultra-small, ultra-flexible probes. (see Gilgunn P J, “An ultra-compliant, scalable neural probe with molded biodissolvable delivery vehicle,” 2012 IEEE 25th International Conference on Micro Electro Mechanical Systems (MEMS), pp. 56-59; DOI:10.1109/MEMSYS.2012.6170092, which is hereby incorporated by reference in its entirety).

In some embodiments, light with wavelengths between 400 nm and 2,000 nm is produced at a light source. The light can be projected through a light source/micro-waveguide 730 onto tissue 720 in the direction of opposing electrode 102. Light from light source micro-waveguide 730 can be received through implanted micro-waveguide 714 and channeled to electrode body 102 to produce the photovoltaic and/or photothermal effect, thereby stimulating tissue 720. In some examples, electrode body 102 and implanted micro-waveguide 714 are coated with a coating as discussed above.

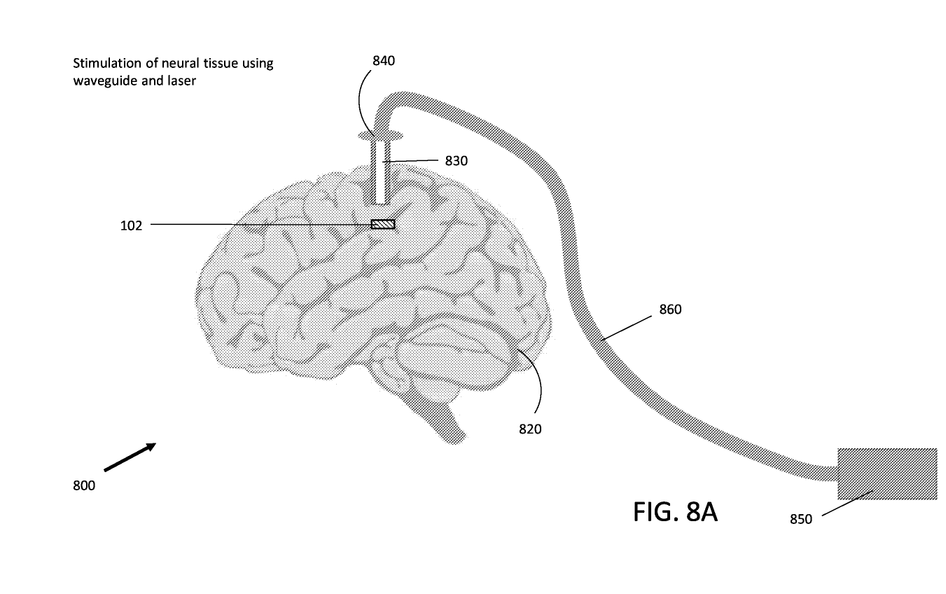

With reference to FIGS. 8A-B, an exemplary system 800 of stimulating neuronal tissue is shown. Many lasers are very large and cannot be mounted to the skull. In some embodiments, light, therefore, is coupled to a head mount chamber using an optical micro-waveguide 830. In some examples, the optical micro-waveguide 830 is coupled to a piezoelectric micromotor 840 to deflect the micro-waveguide and enable rastering over a large brain region. Piezoelectric micromotor 840 allows focusing of light to different portions of tissue 820 without moving micro-waveguide 830. In some examples, focusing can be achieved by mechanically deflecting micro-waveguide 830 by bending. This combination enables high speed specific sequential activation of an array of photovoltatic cell electrodes for patterned activation. While the micro-waveguide may be tethered to the skull, it is uncoupled from the free floating photovoltaic and/or photothermal cell electrode 102. It is contemplated that cell electrode 102 may be coated as described above.

In some embodiments, a subcellular sized micro-waveguide 830 with a diameter between 5 microns and 10 microns, such as 7 microns, is mounted on a surface modified insertion shuttle (for example, see the methods of Kozai T D and Kipke D R (“Insertion shuttle with carboxyl terminated self-assembled monolayer coatings for implanting flexible polymer neural probes in the brain,” J Neurosci Methods. 2009 Nov. 15; 184(2):199-205. doi: 10.1016/j.jneumeth.2009.08.002. Epub 2009 Aug. 8, which is hereby incorporated by reference in its entirety)). The micro-waveguide 840 can then be free floating and fully implanted below the surface of the brain 820 (sub-meningially), and can also be used to enhance photon transfer through the tissue.

With reference to FIG. 8A, electrode 102 is implanted in brain tissue 820. Micro-waveguide 830 is inserted into brain tissue 820, such that it is between 0 and 5 centimeters (cm), such as between 1 and 4 cm, 1 and 3 cm, including 1 cm, 2 cm, 3 cm, 4 cm and 5 cm, from electrode 102. In some examples, a micro-waveguide is inserted into brain tissue and is between 0 and 10 cm, such as between 1 and 9 cm, 2 and 7 cm, including 1 cm, 2 cm, 3 cm, 4 cm, 5 cm, 6 cm, 7 cm, 8 cm, 9 cm, or 10 cm, from an electrode. This proximity allows laser light to impact electrode 102 and induce a voltage to stimulate brain tissue 820. In some examples, laser light, with wavelengths between 400 nm and 2,000 nm, such as between 473 nm and 1040 nm, including 475 nm, 638 nm, and 785 nm, is generated from a laser 850, such as a coherent ultrafast laser. In some examples, average power output of the laser light is between 0.1 mW and 20 mW, including 0.1, 0.2, 0.5, 1, 2, 5, 10 and 20 mW. In some examples, laser pulse duration is between 5 ms and 20 ms, including 5, 10 and 20 ms. In some examples, longer pulse durations are desired to manipulate the membrane potential of nearby tissue (seconds, minutes, hours). In some examples, pulse repetition rate is between 1 Hz and 1,000 Hz, such between 2 and 500 Hz, 3 and 400 Hz, including 4, 10, 30, 50, 150, and 380 Hz. In some embodiments, pulse repetition rates up to 100 seconds, such as up to 10 seconds of MHz to 100s of GHz, are used in applications, such as photothermal therapy of cancers or electroporation. In some embodiments, pulse repetition rate is below 1 Hz, for applications such as for heating purposes as well as low-grade changes in the membrane potential. In some embodiments, laser light is propagated from laser 850 though an optical fiber 860 to micro-waveguide 830. A piezoelectric micromotor 840 can be coupled to micro-waveguide 830 to cause it to deflect, thereby steering laser light to different areas of tissue 820.

In some embodiments, a second electrode (not shown) is implanted at a desired distance from the first electrode 802, such as 0 microns (stacked beside or on top of the first electrode 802) or to a maximum distance in a body. Each electrode can be activated by the same or different wavelengths of light. In some examples, multiple lasers are coupled to a multi-mode fiber micro-waveguide to combine different wavelengths of light and steered using piezoelectric micromotor 840. In some examples the micromotor can deflect the micro-waveguide up to 1 cm. The deflected micro-waveguide can, thus, focus light to different areas of tissue, with a cross-sectional diameter depending on the depth. In some examples, two electrodes are next to each other with different coatings such that the first wavelength of light activates the first electrode; the second wavelength activates the second electrode; and the third wavelength activates neither, or both electrode. Each electrode can, thus, be activated separately or simultaneously to stimulate different neuronal tissue.

With reference to FIG. 8B, an embodiment with a micro-waveguide 830 outside of tissue 820 is shown. In some examples, micro-waveguide 830 is deflected by piezoelectric micromotor 840. Micro-waveguide 830 can touch tissue 820 or it can be located a short distance from tissue 820, depending upon the power of light source 850 and efficiency of optical fiber 860.

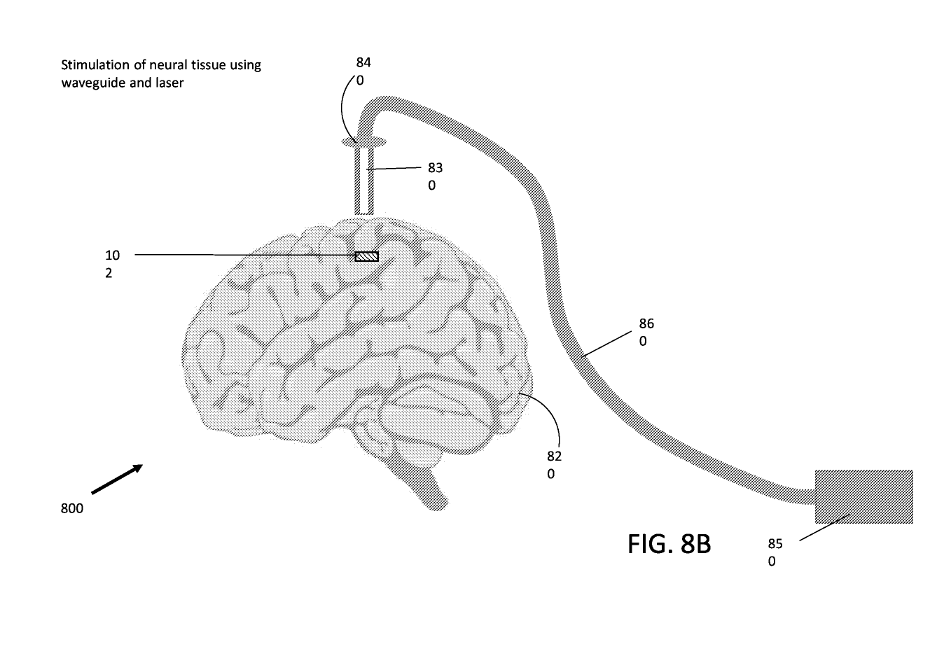

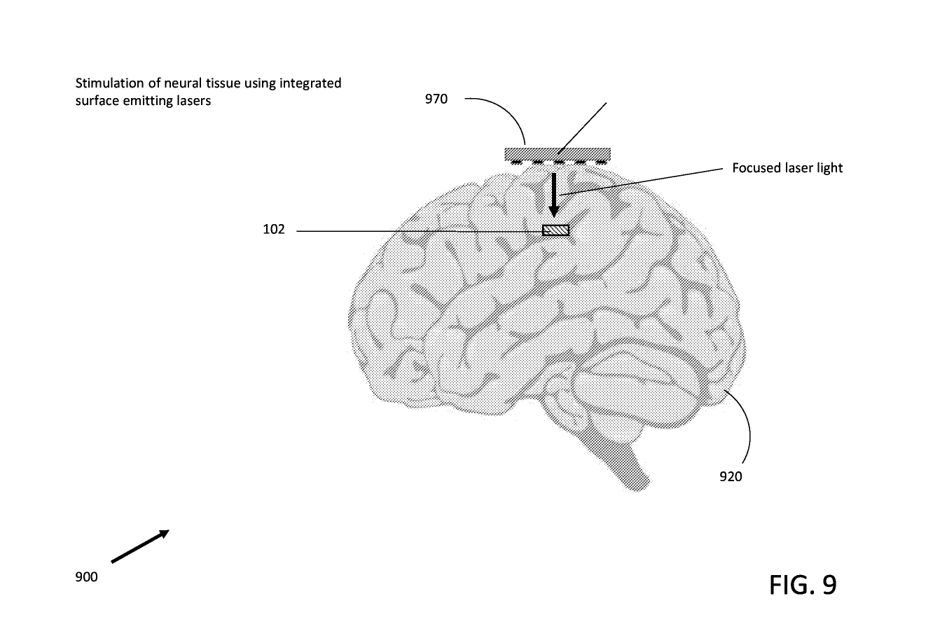

With reference to FIG. 9, an exemplary system 900 of stimulating neuronal tissue is shown. In some embodiments, vertical cavity surface emitting lasers (VCSEL) 970 emits laser light at a wavelength sufficient to activate microelectrode 902. In some examples, microelectrode 102 is implanted, using techniques described herein, in neuronal tissue 920, such as brain tissue or other tissue capable of being stimulated. In some examples, VCSEL 970 comprises a plurality of surface lasers emitting laser light at different wavelengths. In some examples, microelectrode 102 is configured to be activated by a specific wavelength of laser light, which is emitted by VCEL 970.

In some embodiments, at least one other microelectrode is implanted in tissue 920 at some distance from microelectrode 102. VCEL 970 is configured to emit laser light tuned to activation the wavelength for the second microelectrode.

Using these methods, unthethered electrodes induce significantly reduced tissue reaction, and stab wound injury does not significantly impact chronic neural health or glial activation.

V. Wireless Drug Release System

Peripheral nerves provide a relatively easy access to electrical signaling pathways that are involved in a breadth of neurological and physiological conditions such as cardiac hypertension, rheumatoid arthritis, chronic pain, and chronic depression. Hence, the peripheral nervous system (PNS) offers access points to brain regions and end organ targets that can enhance or attenuate neural signaling and hormone, steroid, or cytokine production. For certain conditions, tapping into the PNS provide a promising alternative to traditional systemic pharmacological approaches which can lead to a broad range of side effects.

Current PNS neuromodulation technologies have severe limitations that prevent them from being commonly employed as treatment options over pharmaceutical treatments that have a wide range of side-effects. When PNS devices are prescribed, the relatively large size of these devices requires patients to undergo invasive surgery. The resulting implant usually has poor efficacy and longevity making it a relatively risky value proposition. Moreover, state-of-the-art PNS electrodes such as nerve cuffs need to be large enough to minimize cracking of thin metal electrical traces and abiotic failure. These modes of failure have been observed in the brain, where tissue movement is relatively muted. In contrast to the periphery, it is desirable for the entire device to not only withstand this micromotion, but to have the durability to endure macromotion and traumatic impact from everyday movement. As a result of the increased size, they cause more strain and damage to the tissue. Further, the devices are stiffer [stiffness=(cross-section area)/(length×elastic modulus)] further causing additional mechanical mismatch-related tissue strain and require complex surgery for installation.

Moreover, large stimulation electrode sites lead to broad stimulation of tightly bundled axons. This is further complicated by the fact that large stimulation electrodes are needed to prevent faradaic charge transfer damage to the electrode and tissue. The charge distribution upon stimulation are greatest along the edges of electrode site (because like charges repel), leading to broad preferential activation of excitable tissue along the circumference of the electrode site. A broad challenge with PNS neuromodulation system is developing ultra-small and flexible stimulators and sensors that are also sufficiently strong to withstand movement-related mechanical strains and possess both spatial and chemical species selectivity.

Disclosed herein is a minimally invasive closed-loop neuromodulation system that has improved efficacy and selectivity using highly-stable carbon based technologies. Carbon nano tubes (CNTs) are among the mechanically strongest and high conductive materials currently available, with charge injection limits reported up to 3 mC/cm². Impedance spectroscopy analysis has shown that CNT electrodes have nearly an order of magnitude higher capacitive charge transfer capacity, as well as over eight orders of magnitude greater resistance than traditional metals, which minimize unwanted faradic reactions. Additionally, hollow CNTs allow ions to flow into the tubes which drastically increase the surface area for the electrode's double layer capacitance. This makes carbon a desirable material for PNS sensors. Furthermore, carbon can possess strong photoabsorption from ultraviolet to infrared wavelengths, high power conversion efficiency, high carrier mobility, high thermal conduction, and reduced carrier transport scattering making them an ideal material for scavenging and converting photons for wireless activation of excitable tissue or light-coupled drug delivery. Thus the disclosed system provides for closed-looped PNS or CNS neuromodulation and electrical prescription systems as well as advanced tools for increasing our knowledge of physiological mechanisms of neuromodulation. stimulating, neuro-/bio-chemical modulating, recording, and chemically sensing PNS/CNS interfaces that: i) are ultra-small (sub-cellular), ii) have sophisticated high-sensitivity (efficient energy conversion), high-selectivity (modulating a small volume of neurons (5-200 micron diameter)), high-capacity (0.1 ng to 10 mg of drug release/pulse), and high-performance surfaces(biocompatible, with efficient energy transfer properties and surface area), tailored for specific biological processes, iii) uncouple the mechanical attributes desirable to insert the probe from those that are desirable for long-term implants, iv) are optically selective to specific wavelengths and compatible with existing optogenetic techniques. For example, in some embodiments, the disclosed system has an at least two order of magnitude improvement in device size while maintaining improved strength and compliance over existing state-of-the-art electrodes. The reduced size further leads to an improved selectivity to near single-neuron interfaces. In some examples, a disclosed wireless photoconverter stimulator leads to greater than two-orders of magnitude improvement of spatial selectivity for neurostimulation. Furthermore, because the system utilizes base material made from carbon, it is compatible with MRI systems and resistant to electromagnetic pulse (EMP) attacks.

In some examples, a wired and wireless local drug delivery system is disclosed that regenerates its drug reservoir by scavenging molecules from the environment. In some examples, each stimulus pulse will yield >100 ng (0.1 ng-10 mg) of drug release. In some examples, the system is used for repeatable local drug delivery chronically in vivo. For instance, each stimulus pulse will continue to yield >250 ng of drug release after 300 pulses.

In some examples, the system disclosed herein utilizes a molecular imprinting (MI) technique to render the conducting polymer film ability to self-refill with abundant endogenous molecules (neurotransmitters, cytokines) or recently released molecules (drug, modulators, agonist/antagonists etc.). Molecular imprinting renders polymers the capability of molecule-specific recognition. For example, a polymer is synthesized in the presence of a template molecule, e.g., a target molecule to be recognized or something with similar structure. During the synthesis, monomers organize themselves around the template molecules and form covalent or non-covalent bonds. These three dimensional structures are preserved as the high molecular weight polymer or cross-linked network forms. The template molecules are then removed in some way and leaves behind in the polymers “cavities” having size, shape, orientation and charge distribution complimentary to the template. These cavities can then bind the target molecules with exceptionally high selectivity.

The molecularly imprinted polymer film combined with appropriate electrical control provides a mechanism for recycle of the released molecules or uptake of the endogenous molecules. Released neurochemicals have two fates after being “pumped out”. Some bind to the cell surface receptors and performs their bioactive function; others linger around for a while and diffuse away. Most of these molecules interact with their receptors through affinity binding, which is concentration dependent. When the local concentration decreases, they will become unbound and diffuse away. With molecular printing and proper design of electrical stimulation pulses, these molecules are retrieved before they diffuse away. Likewise, if the molecules to be delivered are various neurochemicals and cytokines naturally produced by the body, molecularly imprinted films will automatically scavenge them at their peak concentration. Such a mechanism can offer a more sustainable source of molecules for chronically implanted wireless controlled release system.

In addition, the electrical control signals for drug release and uptake can be delivered via photovoltaic mechanism as described herein to enable wireless control. The combination of nanostructured conducting polymer, molecular imprinting and wireless stimulation offers a powerful innovation for fully implantable, closed-loop and chronic chemical delivery system that was previously unattainable.

In some examples, a disclosed wireless drug release system is a discrete controlled wireless drug delivery system using conductive polymers with drug containing reservoirs. In addition, this self-replenishing wireless drug delivery system enables repeated chronic drug delivery to a highly localized area, minimizing systemic side effects. Imprinted conductive polymers are developed using select molecules (such as, but not limited to, neuromodulators: GABA, dopamine, and Acetylcholine, anti-inflammatory cytokines: IL-10, pro-inflammatory cytokine receptor antagonists: Kynurenic acid, L-phenylalanine, TGFβ, lipoxin, soluble TNFR, and IL-1RA, and other relevant molecules: adrenaline, glucose, insulin). The conductive polymers scavenge target molecules from the system to be released in bulk when photoexcited, and then regenerate by scavenging the target molecules when photoexcitation is released. In some examples, the disclosed implants can be injected through a syringe IV, SC (even as a tattoo), or endoscopically, or even guided to their final implant position and released using an electromagnetic shuttle.

In some examples, minimally invasive carbon fiber electrical feedback sensors are implanted or injected subcutaneously, intravenously, intramuscularly, or endoscopically to target end organs. In addition, the devices make ideal subcutaneous or intravascular sensors for closed-loop feedback monitoring of activation of wireless stimulators and drug delivery implants.

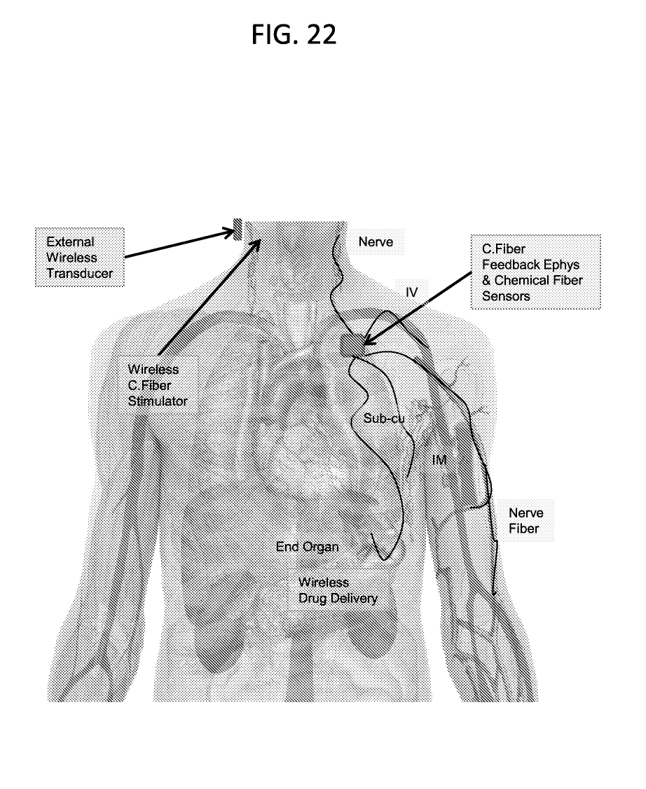

In some examples, chemical sensing fiber technology will also be employed for closed-loop chemical sensing to monitor patient health. This platform technology can be used to monitor chemicals, such as, but not limited to, glucose, dopamine, acetylcholine, catecholamines, oxygen, pH, inflammatory cytokines (IL-18, IL-6), and anti-inflammatory cytokines (IL-10) subcutaneously, intramuscularly, intravascularly, or in/along peripheral nerves and feedback patient condition. In some examples, chemical sensing of select molecules is achieved using high-sensitivity graphene coatings embedded with imprinted polymers and aptamers for sub-second resolution sensing. An advantage of this arrangement is that the feedback sensors do not need to be invasively implanted directly into the nerves to detect meaningful feedback information. Instead, feedback micro-thread fiber sensors are installed through minimally invasive implantation means (injection: IV, IM, SC). Combined, these technologies demonstrate closed-looped peripheral nervous system (PNS) neuromodulation and electrical prescription systems (see FIG. 22) as well as advanced tools for increasing our knowledge of physiological mechanisms of neuromodulation.

In some examples, it is contemplated that a disclosed system could be used to monitor a subject's health condition, such as detect potential to develop or to be afflicted with a particular condition/disease. For example, a disclosed system can be used to monitor or predict soldiers' and civilian patients' health conditions and then treat or pro-actively prevent problems with an automatic hands-free intervention system. For example, an intravascular or subcutaneous sensor (microfluidic or other technology) can be used to detect a particular condition, (e.g., S100B or Interleukin(IL)-1, IL-6, or IL-18 can be used as a marker of traumatic brain injuries or fatal levels of pro-inflammatory cytokines). The system can then automatically wirelessly stimulate the release or upregulation of a particular substance or substances, such as, IL-10, glucose, adrenaline, and/or Kynurenic acid to provide an immediate neuroprotective treatment, analgesic benefit and/or anti-inflammatory cytokine. This automatic and hands-free system can provide an immediate first line of defense even when the subject falls unconscious, and can otherwise eliminate the subject from the dilemma of choosing between focusing on self-treatment or an immediate secondary threat.

In some examples, the disclosed system is utilized to modulate pain. Peripheral targets for pain relief include, but are not limited to, median, ulnar, radial, axilary, suprascapular, brachial plexus, dorsal root ganglia, lateral femoral cutaneous, saphenous, sural, peroneal, tibial, sciatic and femoral nerves. A disclosed system can be characterized for treatment of pain by utilizing one or more acceptable animal models for pain characterization. The three most common models of pain in rat are chronic constriction injury, partial sciatic ligation, and the spinal nerve ligation, and the four most common behavioral quantification assays include heat-hyperalgesia, mechano-hyperalgesia, mechano-allodynia, and cold-allodynia.

i. Conducting Polymers: It is contemplated that the disclosed system can utilize conducting polymers which are a class of polymers with conjugated backbone, which can be charged or neutral depending on the oxidation state. Some conducting polymers can be directly deposited on an electrode surface. As the monomers grow into polymers, the latter are deposited onto the anode, and anions in the solution will be incorporated. This method allows bioactive molecules to be incorporated onto the conducting polymer electrode. These bioactive molecules can be immobilized to encourage specific cell/surface interaction, or releasable to regulate the local biochemical environment. In some examples, the disclosed system utilizes nanoporous conducting polymer polyaniline (PANI), polypyrrole (PPy) and/or poly(3,4-ethylenedioxythiophene) (PEDOT).

ii. Loading/Release Mechanisms It is contemplated that both anionic and cationic agents can be incorporated into and electrically released from conducting polymer electrodes. However, the loading and release mechanisms will vary depending upon the type of agent. In some examples, anionic drugs act as dopant molecules that are incorporated into the polymer by electropolymerization. When polymer/drug complex is under a negative potential, the positively charged polymer backbone is reduced and loses charge; as a result, the drug molecules dissociate from the backbone and diffuse out of the film and into the electrolyte solution. For cationic delivery, an anionic polyelectrolyte (such as, poly-styrenesulfonate, a commonly used polyelectrolyte dopant for conducting polymers) can be added. Some of its negative charge will balance the charge on the polypyrrole backbone, while the extra negative charge can be used to bind to the cationic drug. In some examples, drug molecules are loaded during the electropolymerization. In some examples, drug molecules are loaded after polymerization when the polymer is soaked in drug solution under negative electrical potential. The release of the cationic drug also utilizes the redox property of the polymer, but with positive potential as the trigger. This feature allows charged drug molecules to be incorporated into the polymers, which can be repeatedly released in response to electrical stimulus.

iii. Molecular Imprinting. Only a small amount of released molecules find their receptors and bind to them and the rest diffuse away. Instead of letting these molecules unbound and diffuse away, molecular imprinting is used to structure the drug releasing polymer so that it may specifically uptake the released molecules as well as scavenge new molecules from the environment to recharge to drug reservoir. In addition, excitatory and inhibitory neurotransmitter and anti-inflammatory signaling molecules, such as, but not limited to acetylcholine, dopamine, GABA, IL-6, IL-10, Kynurenic acid, L-phenylalanine, TGFβ, lipoxin, soluble TNFR, and IL-1RA can be found in vivo. Ability to actively uptake these molecules offers a more sustainable source of drug and extend the lifetime of the drug-releasing polymers.

Molecularly-templated nanoporous and/or nanofibrous films, such as polypyrrole films, are utilized to target molecules of interest, such as, but not limited to, GABA, acetylcholine, Kynurenic acid, and L-phenylalanine. In some examples, nanostructured films are fabricated and then evaluated for uptake ability of the target molecule. Methods for evaluating uptake include those provided herein including the Example Section. The synthesis conditions can then be optimized to maximize the uptake. Post-synthesis processing conditions including uptake capacity and specificity can also be optimized so that the most complimentary cavities with minimum compromise on other desired properties. In addition to synthesis and post-synthesis processing conditions, uptake may be facilitated by varying electrical potential.

In one particular example, a molecularly imprinted PPy film, nanostructured or not, is synthesized through the same electrochemical polymerization of pyrrole in the presence of the drug molecules as described in herein. To obtain high quality molecularly imprinted polymer films, the synthesis parameters are optimized. Also, the wall material of the nanoporous film or the nanofibers of the nanoporous film is compacted at the molecular scale to be able to memorize the shape of the released drug. Such films are constructed using solutions with high drug/pyrrole ratio with slow polymerization rate. After the drug molecules are incorporated into the polypyrrole film, removal of those molecules leave complementary cavities in the PPy film. To remove the template drug molecules, several conditions are employed. In some examples, a synthesized film is exposed to overoxidization conditions: PPy film is subjected to a positive potential higher than 0.9 V in PBS. In some examples, a synthesized film is exposed to drug releasing conditions such as a constant negative voltage, a cyclic votammetric stimuli or a train of negative pulses. In some examples, a combination of the aforementioned is utilized—electrical stimuli applied alternately with the goal of fully releasing the template while conserving the shape of complimentary cavities. Photoexcitation can be used to lead to conformational change in the conductive polymer matrix leading to the expulsion of the imprinted molecules. Release of the photoexcitation allows the empty cavities to scavenge molecules from the environment.

The following examples are provided to illustrate certain particular features and/or embodiments. These examples should not be construed to limit the invention to the particular features or embodiments described.

### EXAMPLES

### Example 1

This example illustrates the ability to stimulate local neuronal tissue using a disclosed stimulating microelectrode.

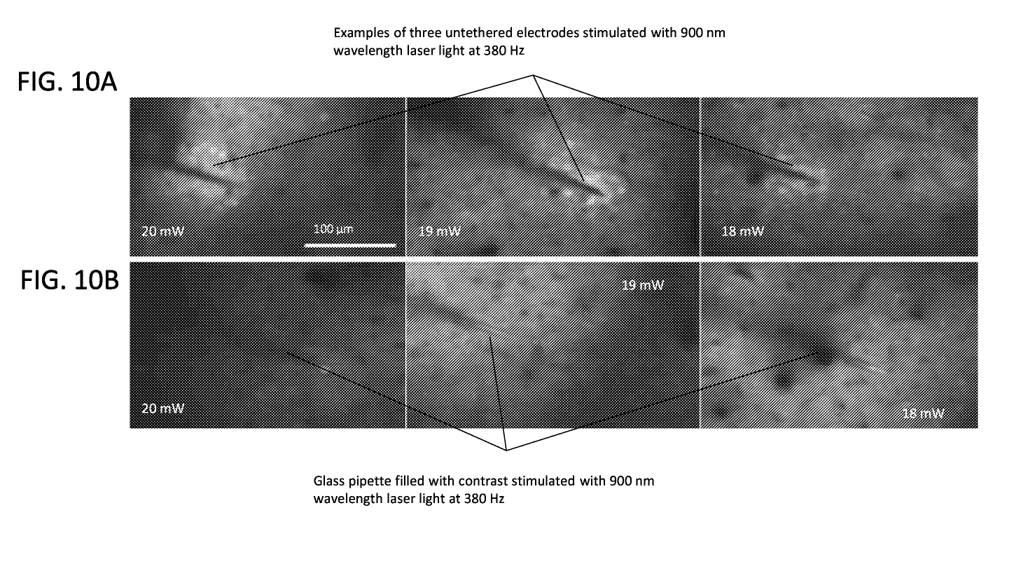

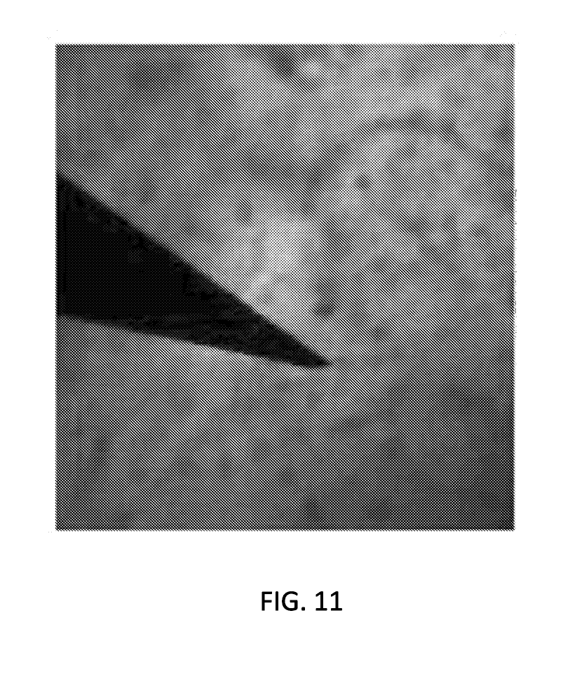

Two-photon images as shown in FIG. 10 were obtained during stimulation of brain tissue using an untethered photoelectric electrode made according to the principles of this disclosure. A 7 μm diameter carbon fiber microthread photovoltaic electrode, insulated with parylene-C, was placed 250 μm below the brain surface in each of three transgenic mice, as shown in FIG. 10A. Control 7 μm diameter glass pipettes are shown in FIG. 10B. The transgenic mouse model expressed GCaMP3 across the cortex including pyramidal neurons. GCaMP3 is a genetically encoded calcium indicator that increases in fluorescence intensity when intracellular calcium changes during neuronal depolarization. The two-photon z-stack image was taken using an optic fiber placed over the surface of the brain at an angle of 30 to deliver the photo-stimulus. To avoid potential stimulation from the imaging laser the area occupied by the photovoltaic electrode was masked, as shown in FIG.11. Activation of neurons is evidenced by an increase in fluorescence in FIG. 10A.

The photovoltaic electrode was stimulated with 900 nm laser light at 380 Hz frequency. Power levels of 20 mW, 19 mW, and 18 mW were used as shown from left to right in FIG. 10. The number of activated neurons, distance of activated neurons from the electrode and percent of fluorescence changes varied with laser intensity. Stimulation with the photovoltaic microelectrodes is shown in FIG. 10A whereas stimulation with glass pipettes is provided in FIG. 10B.

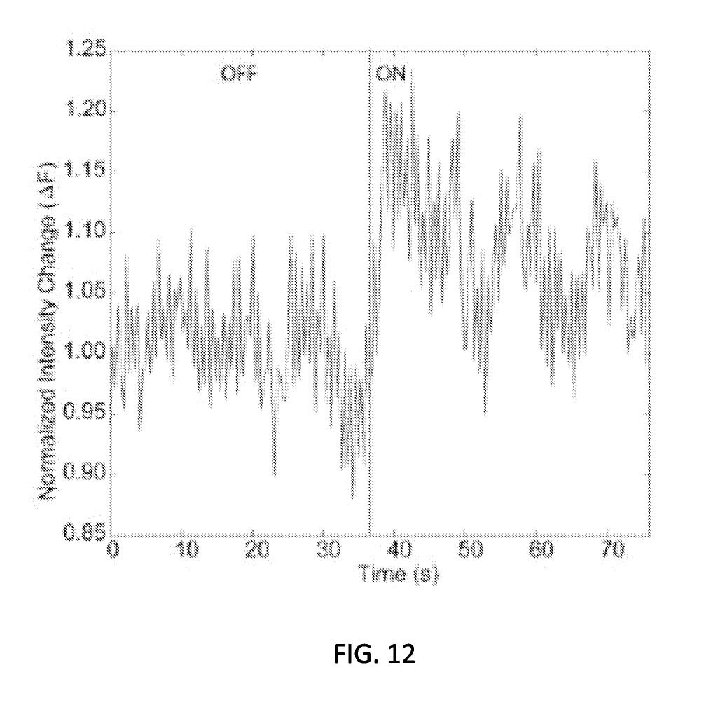

FIG. 12 shows fluorescent intensity of calcium indicator dye labeled neurons next to the photovoltaic and/or photothermal stimulation electrode. The electrode was implanted 250 μm deep in cortical tissue. Fluorescent intensity was recorded using two-photon microscopy. The recording shows fluorescent intensity before and after stimulation with the photovoltaic electrode. To the left side of the line in the middle of the plot, labeled as “OFF,” the stimulating laser is not focused on the electrode. To the right of the line, labeled as “ON,” 920 nm laser light at 150 Hz frequency was focused on the electrode at 20 mW. The plot shows that when the disclosed electrode was excited by laser light, the fluorescent intensity of nearby neuronal tissue increased substantially.

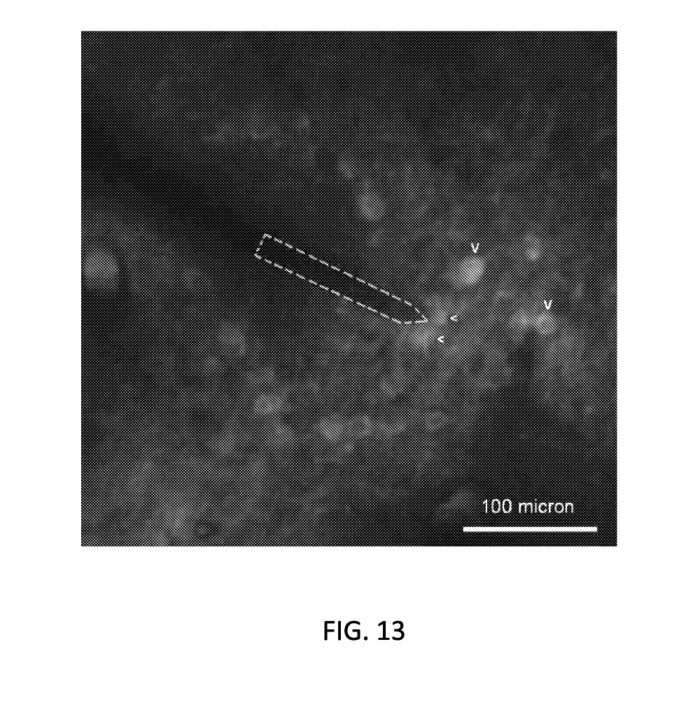

FIG. 12 also demonstrates that photons absorbed onto an implanted untethered 7 μm diameter parylene-C insulated carbon fiber generate electrical voltages sufficient to excite nearby neurons. The microelectrode was stimulated with 900 nm laser light at 150 Hz and 10 mW. The electrode is shown in FIG. 13 by the dashed lines and stimulation of GCaMP3 neurons in mouse brain is indicated by arrows.

These studies indicate that the disclosed electrode is effective in stimulating neuronal tissue when implanted directly in the tissue and is untethered to the stimulating laser.

### Example 2

This example illustrates that the size and geometry of a structure has a direct effect on cellular response.

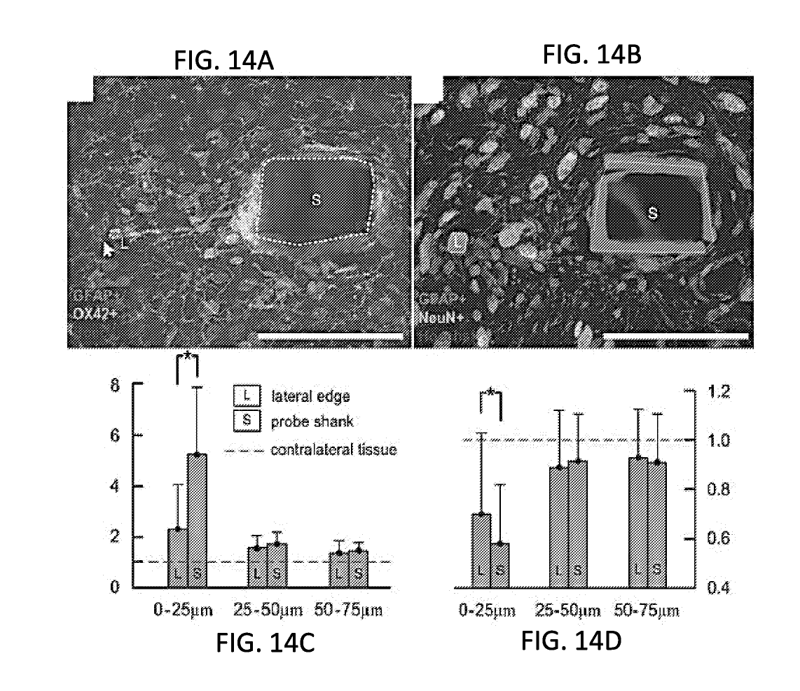

In rat subcutis, fibers with diameters from 2 to 12 μm have a marked decrease in glial encapsulation. Neural probes with sub-cellular dimensions reduce the foreign body response by preventing cellular adhesion or gliosis. Given the strength limitation of such structures, microfabricated, thin polymer structure that attached to a larger, conventional shank was developed. Parylene-substrate neural probes having a stiff penetrating shank, i.e., 48 μm by 68 μm, supporting a thin lateral extension, i.e. 5 μm thick and 100 μm wide, were fabricated (denoted by “S” and “L” in FIG. 14A-B). Probe structures fabricated with a sub-cellular dimension significantly reduced glial scar encapsulation while preserving neuronal populations around the lattice edge.

With reference to FIGS. 14A-14D, examples of qualitative and quantitative results around a nonfunctional edge lattice probe are shown. FIG. 14A shows GFAB and OX42 antibody labeled astrocytes and microglia. FIG. 14B shows GFAB and NeuN labeled astrocytes and neuronal nuclei. FIG. 14C shows the normalized mean nonneuronal density as a function of distance from probe interface. FIG. 14D shows the mean neuronal density. The scale bars for FIGS. 14A and 14B are 100 microns. Significance of FIGS. 14C and 14D is P<0.05.

These studies indicate that the size and geometry of a structure have a direct effect on cellular response.

### Example 3

This example illustrates the use of carbon fiber electrodes to record neuronal signals in brain tissue.

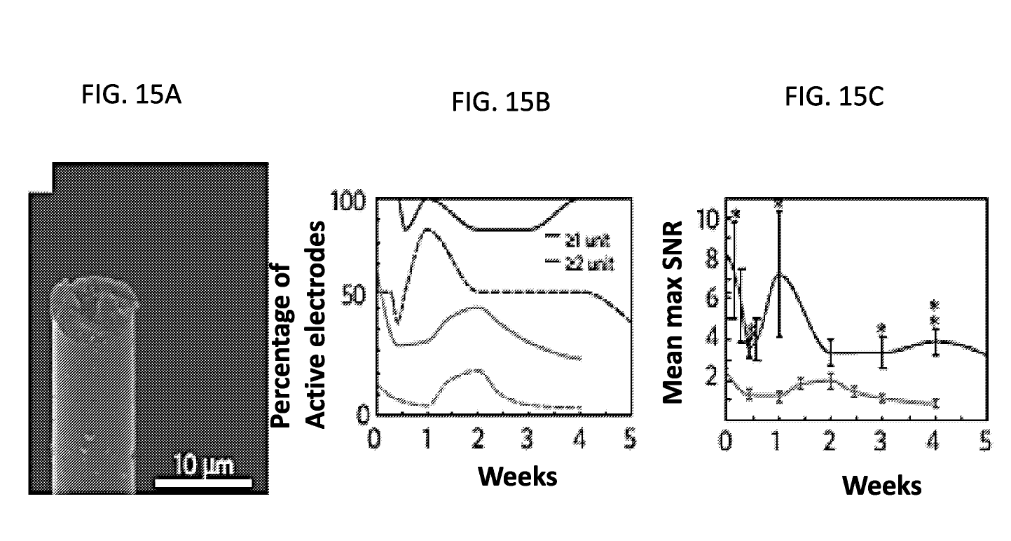

With reference to FIGS. 15A-15C, a 7 μm diameter composite microthread electrode formed of a carbon-fiber core, a parylene-based thin-film dielectric insulator that is chemically functionalized to control intrinsic biological processes, and a PEDOT/PSS recording pad, was demonstrated to record electrophysiological signal more robustly than traditional electrodes.

The resulting implants are an order of magnitude smaller than traditional recording electrodes, and more mechanically compliant. These devices recorded single unit activity from motor cortex with significantly greater amplitude and yield, as well as elicited significantly less BBB damage and reactive tissue response when compared to traditional planar electrodes.

While the sub-cellular sized electrodes induced significantly less chronic reactive tissue response, the tissue response was still greater than unimplanted control. Other studies demonstrated that chronically implanted untethered probes elicited significantly less tissue response. Furthermore, a follow-up study demonstrated that fully implanting probes below the meningial surface of the brain lead to dramatically less reactive tissue response compared to untethered probes implanted conventionally with the upper end penetrating the meninges, but not attached to the skull.

FIG. 15A shows an SEM image of a microthread electrode (MTE). FIG. 15B shows a single-unit yield of MTE (upper traces) vs. silicon Mi probes (lower traces). FIG. 15C shows a single-unit signal-to-noise-ratio of MTE (upper trace) vs. silicon Mi probes (lower trace). Chronic neural spike activity was reported as it is considered a sensitive assay of chronic neural interface.

### Example 4

This example illustrates neuronal binding to microelectrodes coated with a bioactive coating.

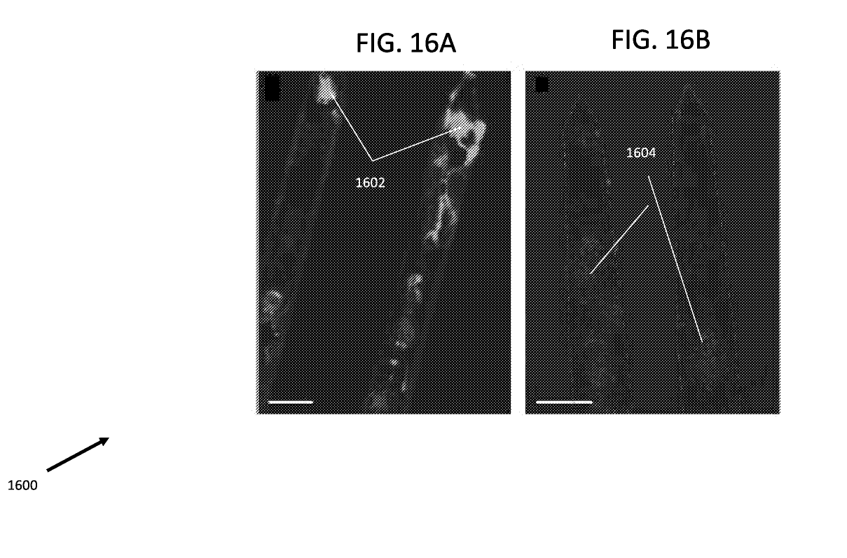

With reference to FIGS. 16A-16B, images 1600 of probes coated with a bioactive coating and uncoated probes are shown. FIG. 16A illustrates neurons 1602 binding to 4 week chronic implants of L1 Cell Adhesion Molecule (L1CAM) coated probes. FIG. 16B shows uncoated probes with no neural binding; dapi 1604 shows substantial non-neuronal cells adhering to uncoated probes. The scale of FIGS. 16A and 16B is 50 μm.

### Example 5

This example illustrates photovoltaic activation of carbon fibers and doped silicon using a tunable laser and methods of testing photovoltaic and/or photothermal microelectrodes.

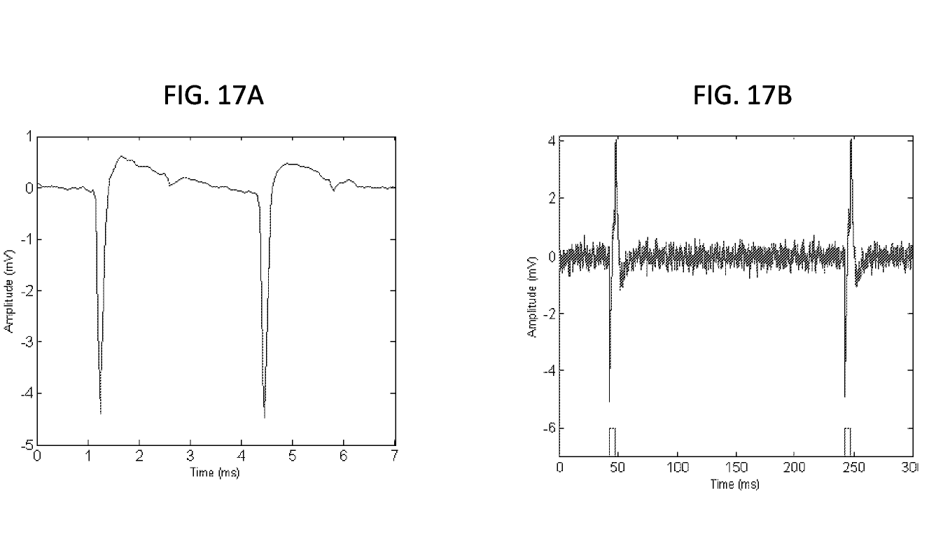

For testing, the microelectrode is placed in a beaker with a CSF solution and 0.5% agarose to restrict movement. An optic fiber is placed 200 μm over the microelectrode using a micromanipulator mounted on a stereotaxic frame and a ground lead can be placed in the beaker as reference. The laser consists of 473, 532, 638, 785 nm laser sources (CrystaLaser) as well as between 690 nm to 1040 nm every decade with a tunable laser (Spectra-Physics). The power and pulse width is modulated and the generated voltage is recorded using an electrophysiology recording system (RX7, Tucker-Davis Technologies) and a potentiostat (Autolab PGSTAT12, Metrohm) using a platinum electrode placed proximal to the photo-converting material, for example, see FIGS. 17A and B. The platinum electrode is chosen because it has the highest work function and limited photoelectric activation (wavelength: <195 nm: see Table 1). Laser light powers of 1, 2, 5, 10 and 20 mW are tested and pulse durations of 2, 5, 10 and 20 ms are delivered every 1 sec.

With reference to FIGS. 17A and 17B, the amplitude in millivolts of voltage induced in photovoltaic microprobes is shown. FIG. 17A shows two-photon photovoltaic activation on a carbon fiber photoconverter using 800 nm, 1 mW laser in 0.5% agarose in saline. No single-photon activation was observed when undoped carbon was stimulated through a single-mode optic fiber.

FIG. 17B shows an electrical signal (upper trace) produced by single-photon activation of a photosensitive n-doped silicon photoconverter using 473 nm, 10 mW laser with 5 ms pulse width (lower trace). Carbon fiber electrode recorded ionic current 100 μm away from photovoltaic converter in 0.5% agarose in saline.

In vitro patch clamp studies using primary neuronal culture to further characterize the kinetics of neuronal activation by short pulse width (ns to fs) and high laser power (high voltage) photovoltaic stimulation may be utilized. Limits defined by electroporation and electrolysis for extracellular and intracellular photovoltaic electrodes define the test space. Electroporation can be examined by monitoring the membrane potential following a pulse train. Electrolysis of water is of limited concern with photovoltaic electrodes due to the preferential non-Faradaic charge transfer, especially when coupled with short pulse widths. In vitro studies with extended photovoltaic stimulation in saline with 30 ms pulse width and 100 mW laser output did not lead to noticeable gas evolution.

### Example 6

This example illustrates the ability to create monolithically integrated micro-waveguides to mix and split laser light.

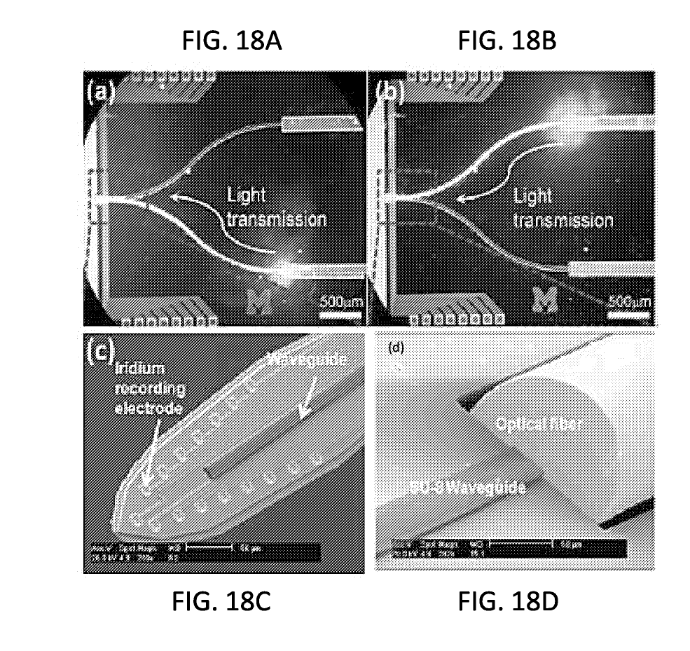

With reference to FIGS. 18A-18D, examples of monolithically integrated micro-waveguides are shown. For methods of fabricating micro-waveguides see Wu F. et al.(“An implantable neural probe with monolithically integrated dielectric waveguide and recording electrodes for optogenetics applications,” J Neural Eng. 2013 October; 10(5):056012. doi: 10.1088/1741-2560/10/5/056012. Epub 2013 Aug. 28, which is hereby incorporated by reference in its entirety). These waveguides enable complex combination or selective wavelength activation of adjacent photovoltaic electrodes. The laser path can also be modified to enhance entanglement of photon pairs (e.g. biphotons). Biphotons delivered through a single-mode fiber further increase efficiency compared to the same intensity of coherent light in the two-photon processes. Because lasers are very large and cannot be mounted to the skull, light can be coupled to a head mount chamber using optical micro-waveguides. These micro-waveguides can be coupled to a piezoelectric micromotor to deflect the micro-waveguide and enable rastering over a large brain region. This combination can enable high speed specific sequential activation of an array of photovoltatic micro-electrodes for patterned activation. While the micro-waveguide may be tethered to the skull, it is uncoupled from the free floating photovoltaic cell electrode. Laser light delivered through microfabricated oxynitride dielectric micro-waveguides has been showed to perform efficiently and has demonstrated improved spatial precision and scalability for multichannel and multi-wavelength photovoltaic stimulation.

FIG. 18A and FIG. 18B show a micro-waveguide mixer with light transmission from two different sources. Laser light is delivered to the same distal end of the micro-waveguide no matter which source is used. FIG. 18C shows a microprobe with integrated iridium recording electrodes and a micro-waveguide in the center. FIG. 18D shows coupling of an optical fiber to an integrated micro-waveguide.

### Example 7

This example illustrates a vertical cavity surface emitting lasers (VCSEL) as a laser light source to active untethered/wireless micro-photovoltaic electrodes and provide for spatial selectivity.

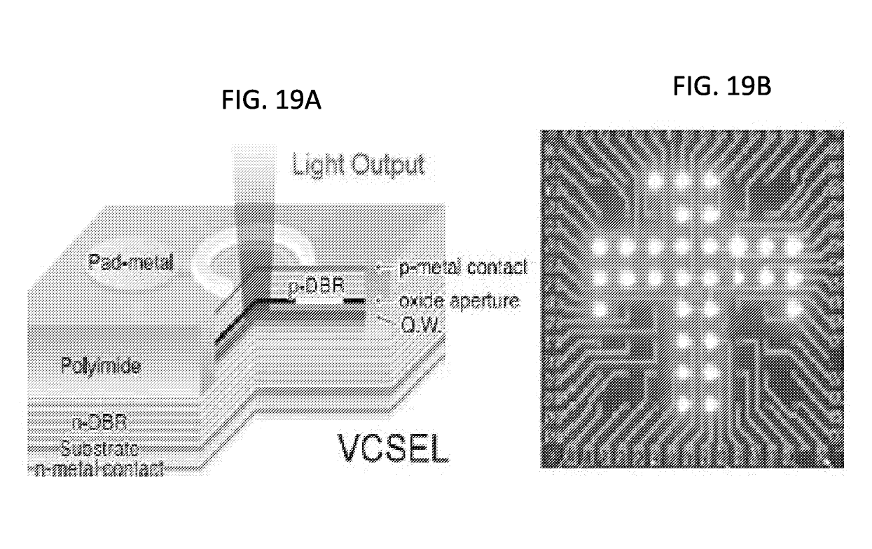

With reference to FIGS. 19A and 19B, exemplary VCSELs are shown. VCSELs have several unique advantages for integration into optical microsystems. First, the power consumption (and hence heat dissipation) is lower than any competing laser technology by at least one order of magnitude, with electrical power consumption on the order of 2 mW (1.1 mA*1.8V) for continuous operation. The low power consumption means that small batteries can be used to drive the VCSELs and the heat dissipation by the VCSELs is minimal In addition, VCSEL lasers have very small dimensions (4 μm) which allows high overall laser powers to be achieved by using tightly packed arrays of lasers. The VCSEL fabrication process is particularly suited to produce 2D arrays of lasers, with pitches down to about 40 μm. Thus, a typical 3-inch diameter GaAs wafer yields more than 10,000 VCSELs on a 0.5-mm pitch and is capable of producing extremely high photon densities (>400 mW/cm̂2).

FIG. 19A shows different layers of a VCSEL during the fabrication process. FIG. 19B shows a fabricated VCSEL with thirty-three surface emitting lasers.

### Example 8

This example illustrates an in vivo longitudinal biocompatibility and stability comparison of disclosed photovoltaic and/or photothermal microelectrodes to state-of-the-art stimulation electrodes.

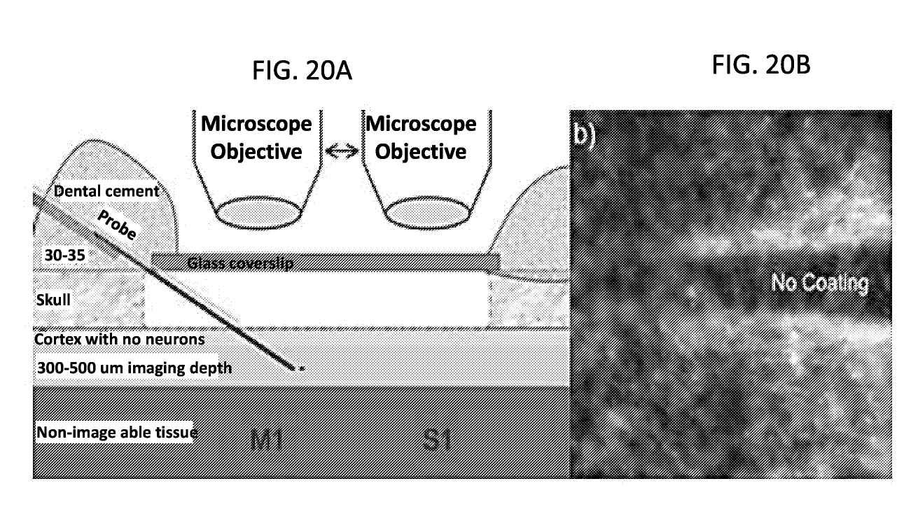

Referring to FIGS. 20A and 20B, Thy1-GCaMP3 mice are implanted with stimulation electrodes into the M1 cortex. A chronic cranial window, probe, and/or optical micro-waveguides are mounted on the skull. Stimulation of the primary motor cortex of can be conducted with the appropriate stimulation mode. Time-locked calcium imaging from the primary somatosensory cortex is used to identify functional activation of feed-forward circuits. Fluorescence intensity in target S1 cortex and M1 cortex can be evaluated for functional stimulation. Electrode position and activated neurons can be examined with respect to neuronal and vascular structures to identify the ability to activate the same discrete population of neurons and identify ‘spatial drifts’ or ‘positional movement’ of the electrodes. Optical stimulation, electrical stimulation, and photovoltaic stimulation can be compared. Photovoltaic stimulation is carried out as previously described. Blood brain barrier (BBB) leakage can be quantified by injecting a red intravascular dye (Sulfarhodamine101; SR101).

Sixteen weeks following surgery, subjects are intracardially perfused with 4% paraformaldehyde. The brain is cryoembedded and cryosectioned horizontally, which is compatible with the immunohistochemistry method selected. In addition to the nuclei counterstain Hoescht, antibodies are used to label for neurons (NeuN), microglia (IBA1), astocyte (GFAP), meningeal fibroblasts (vimentin), blood-brain barrier (tomato plant lectin), neurofilament (NF200), apoptosis (Caspase-3), and blood-brain barrier leakage (IgG and ferritin). Images are collected using a 4 channel confocal microscope.

A linear mixed effects model (ANOVA) is used to test average number, distance, and density of activated neurons, changes in calcium intensity fluorescence, neuronal density, glial scar, apoptosis, and nonneuronal density between each stimulation mode. For each metric, one-way standard ANOVA is used to compare the difference between coatings, followed by Tukey's Honest Significant Difference (HSD) post-hoc to identify pairwise differences. Differences are considered significant for p<0.05.

If interference between in vivo GCaMP imaging and stimulation electrodes is significant, the chronic stability can be evaluated by chronic electrophysiology in non-imaging animals. Single-unit stability and multi-unit firing rate changes can be evaluated using microelectrode arrays implanted into the feed-forward S1 cortex.

### Example 9

### Use of Transgenic Mouse Model for Characterization Studies

This example illustrates use of a transgenic mouse model to characterize and evaluate the disclosed stimulating electrodes.

A transgenic mouse model expressing GCamp3 across the cortex including layer pyramidal neurons is used to evaluate and characterize the disclosed stimulating electrodes. GCamp3 is a genetically encoded calcium indicator that increases in fluorescence intensity when the intra-cellular calcium changes during neuronal depolarization. Benchmark studies compare the performance of photovoltaic electrodes and standard stimulation electrodes in GCamp3 mice as well as a channelrhodopsin-2 (ChR2) mouse model consisting of GCamp3 expressing ChR2 using AAV virus. Typical group size is N=8-10. Performance is characterized in terms of the stimulated spatial selectivity, density, and changes in intensity. Initial pilot studies determine the reliability and durability of the following photovoltaic stimulation laser coupling mode: fixed position optical fiber, rastering optical fiber with piezomotor, microfabricated optical waveguide, or VCSEL.

GCamp3 transgenic mice (B6.CBA-Tg(Thy1-GCaMP3)6Gfng) are obtained from Jackson Laboratories (Bar Harbor, Me.) and anesthetized using ketamine-xylazine for surgery. A craniotomy is performed over the primary motor cortex (M1) and a well is placed to reinstate intracranial pressure. 3D two-photon vascular mapping is used to avoid insertion through major intracortical blood vessels using methods previously developed (see Kozai T D, et al., “Reduction of neurovascular damage resulting from microelectrode insertion into the cerebral cortex using in vivo two-photon mapping,” J Neural Eng. 2010 August; 7(4):046011. doi: 10.1088/1741-2560/7/4/046011. Epub 2010 Jul. 19, which is hereby incorporated by reference in its entirety). A photovoltaic electrode is inserted to a depth of 200 μm under the pial surface. A two-photon z-stack image is taken from Thy1-GCaMP3 mice cortex. An optic fiber is placed over the surface of the brain at an angle of 30 degrees to deliver the photo-stimulus. To avoid potential photo-stimulation from the imaging laser, a mask, as shown in FIG. 11, is applied to the scanning laser over the location of the electrodes such that the laser will be blanked (or zeroed) once it reaches the vicinity of the electrode.

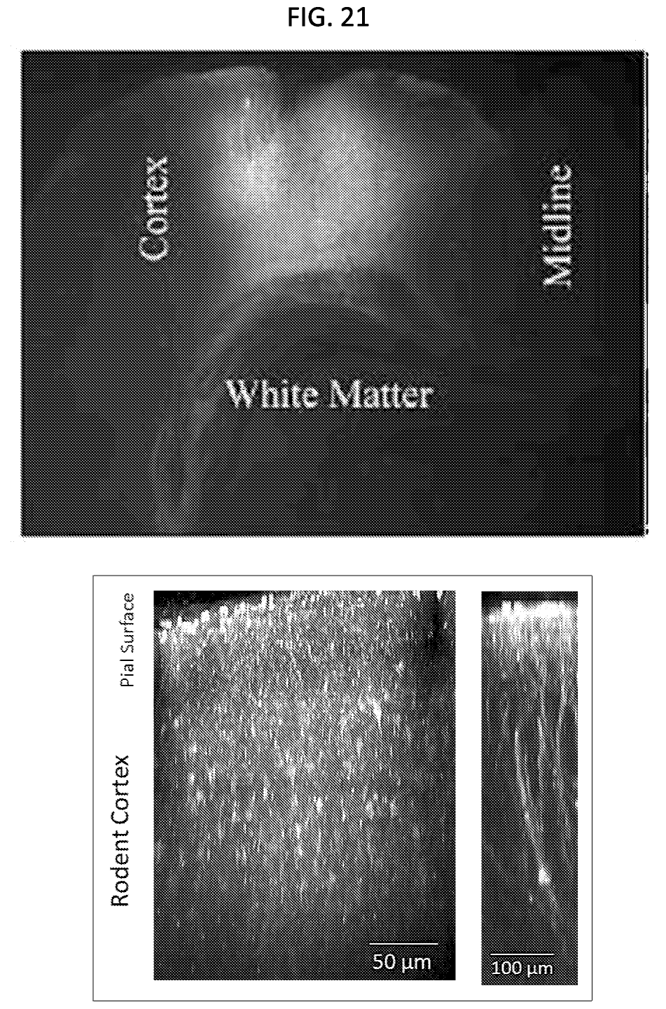

With reference to FIG. 21, the mice to be used can also require transduction of optogenetic proteins by means such as an injection of AAV performed 3-4 weeks prior to the study to drive the expression of ChR2 (e.g., AAV-hSyn-ChR2; see methods described by Lowery R L, Majewska A K, “Intracranial injection of adeno-associated viral vectors,” J Vis Exp. 2010 Nov. 17; (45). pii: 2140. doi: 10.3791/2140, which is hereby incorporated by reference in its entirety; and Lowery R L, et al., “Rapid, long-term labeling of cells in the developing and adult rodent visual cortex using double-stranded adeno-associated viral vectors,” Dev Neurobiol. 2009 Sep. 1; 69(10):674-88. doi: 10.1002/dneu.20735—which is hereby incorporated by reference in its entirety).

### Example 10

### Characterization of Disclosed Wireless Regenerating Drug-Delivery System

This example illustrates an exemplary system which wirelessly employs chronic neurochemical modulation of peripheral nerves and at end organs.

Current drug delivery devices are bulky in size and have poor spatial and temporal resolution preventing the targeting of individual axons or nerve endings in a close-looped manner. Nanostructured conducting polymer based drug delivery systems allow electrically controlled release from microdevices. The electrical control provides a possibility for on-demand close loop drug delivery. However, the drug loading capacities are limited by the thickness and size of the polymer film and the system cannot be easily replenished. Microchannel based drug delivery systems may allow drug refill but utilize connections to external reservoir which increase the complexity and size of the implant as well as chance of infection. The ability to increase the longevity of the drug delivery capabilities without increasing implant size, combined with highly controlled repeatable local drug delivery system without transcutaneous or percutaneous tubing is desirable for allowing inhibiting or priming nerves as well as chemical/physiological modulation of other biological processes such as attention, adrenaline, healing, and systemic inflammation/injury response.

Nanoporous polypyrrole (PPy) films incorporated with drugs (Fluorescein) were prepared using PS nanobeads as templates according to the method described in (Luo et al., Chemistry 13, 2138-2143 (2007), which is hereby incorporated by reference in its entirety). The drug release properties of these nanoporous films in phosphate buffer solutions (PBS) were characterized. The amount of drug release triggered but the electrical pulses from the nanoporous PPy film was about 8 times more than that from the normal PPy film. Without the electrical stimuli, there is a negligible amount of drug diffusion. This result is consistent with the belief that nanoporous film are able to load more and release more drugs due to the high surface area.

An overoxidation condition has been used in previous studies to obtain molecularly imprinted polypyrrole (Syritski et al., Electrochimica Acta 53, 2729-2736 (2008) which is hereby incorporated by reference in its entirety. Overoxidation conditions were applied to the nanoporous PPy/flurorescein films and then a −2V stimulus was used to drive out most of the drug. The resulting film was placed in flurorescein solution and the uptake of flurorescein was measured by quantifying the concentration change of the solution. The molecularly imprinted nanoporous PPy film took in about ⅓ of the drugs it released. In contrast, the film that had not been subjected to electrical stimuli for drug release did not have any uptake. This study indicates that released molecules may have left cavities in the film and these cavities have high affinity to the released molecule. The drug uptake ability may be further improved after optimization of the preparation and/or with the application of a suitable electrical potential to facilitate the uptake as disclosed herein.

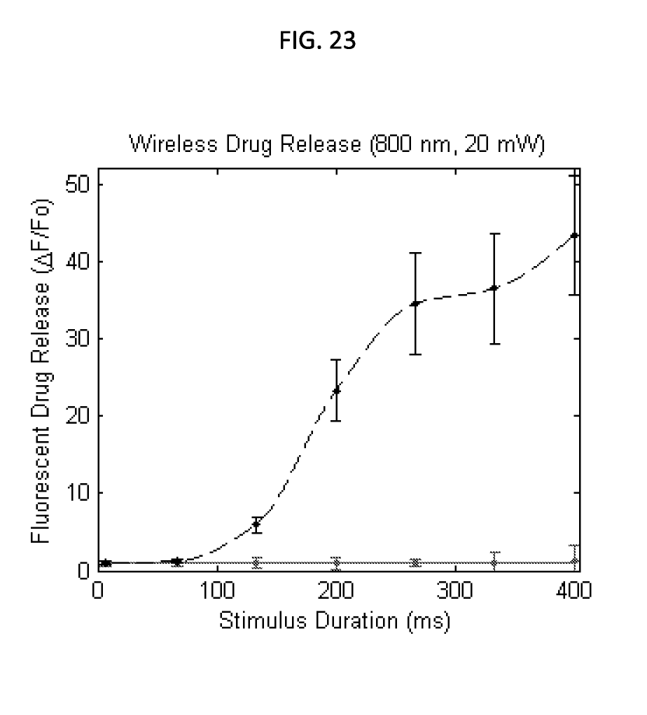

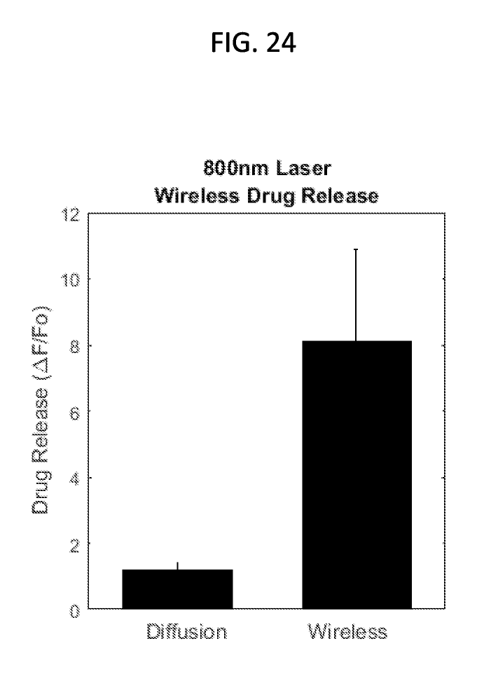

In further, these coatings were applied to photoconverters and laser stimulation was used to release drugs from the photoconverters (FIGS. 23 and 24). The released amount was consistent with the amount released from wired electrical stimulation.

### Example 11

### Electrically Controlled Dexamethasone Release System

This example demonstrates electrically controlled dexamethasone release system based on the conducting polymers polypyrrole (PPy) or PEDOT(Kolarcik et al. Journal of Neural Engineering. 2015, 12(1). 016008; Alba et al. Biosensors. 2015, 5(4), 618-646; Luo et al. Biomaterials. 2011. 32. 6316-6323; Luo et al. Electrochemistry Communications. 2009, 11, 1956-1959; each of which is hereby incorporated by reference in its entirety). Dexamethasone sodium phosphate (Dex) was incorporated into PPy released in response to a cyclic voltammetric (CV) stimulus. In contrast to some previous studies that use step potential stimuli, the CV stimulation can produce a true linear drug release vs. the number of stimuli. These results show that from a PPy/Dex film, 0.5 μg/cm² Dex per stimulus can be released and up to ˜15 μg/cm² after 30 CVs. After 30 episodes of stimulated release, the PPy polymer film begins to show signs of depletion. Using cultured murine cerebellar astrocytes, the electrically-released drug was as effective in reducing astrocyte counts as drug that is directly added into the culture. Also, focal release of neurochemicals in conjunction with multi-channel recording and stimulation can be used to modulate the network dynamics in vitroand in vivo.

### Example 12

### Characterizing a Prepared Electrode

This example provides methods for characterizing a prepared electrode. A prepared electrode can be characterized to determine one or more of the following: (1) drug-loading capacity; (2) drug-releasing efficiency (speed and consistency); (3) electrical impedance of coated electrodes; (4) chemical and electrical stability of the polymer-drug complex. In some examples, to measure drug release, coated electrodes are immersed in 1 ml of PBS solution and subjected to electrical release stimuli. After each stimulus, the concentration of the released drug in the solution is quantified using different analytical tools, such as fluorometric assay and OPA (o-phthaldialdehyde) derivatization, and fluorometry is used to quantify the released molecules. To evaluate the dynamic of drug release, a real time imaging system can be used to visualize the dye release from the microelectrodes and semi-quantitatively characterize the release profile. An inverted fluorescence microscope can be used to provide a qualitative characterization. In some examples, an upright confocal microscope is utilized for quantification. Evaluation of standard solutions of dye can be performed using the confocal microscope to determine the optimum optical and detection parameters for high-resolution dye release imaging, and to prepare a calibration spectrum for the quantitative assessment of dye concentration using fluorescent luminescence. Electrically stimulated release of dye from multi-electrode arrays can be evaluated using the confocal imaging system, with high-resolution images captured at high speed above the electrode surface over the entire duration of the release stimulus. Fluorescence intensity from a fixed-thickness layer can be used to evaluate dye concentration above the electrode, which will be interpreted to study dye release uniformity, density, and kinetics, as well as dye diffusion away from and towards (in the case of uptake) the electrode surface at various time points during the stimulus process. This allows for both a visual and quantitative assessment of the real-time dynamics of dye release from the conducting polymer system, and for improved optimization of the release methodology and protocol. Electrical impedance of an electrode coated with the conducting polymer-drug film before and after drug release can be characterized using impedance spectroscopy. Electrical, chemical and mechanical stability of the polymer-drug film in the culture media, before and after repetitive release, will be characterized using cyclic voltammetry, Fourier Transform Infrared Spectroscopy, and surface analyses such as optical microscopy, scanning electron microscopy and atomic force microscopy.

### Example 13

### Synthesis of High-Capacity Reservoir Drug Delivery Coating

This example describes an exemplary synthesis for nanoporous, nanofibrous and nano-drug reservoir based polymer film coating.

i. Nanoporous. To synthesize nanoporous PPy, self-assembled multilayer PS nanobeads are used as templates. The PS templates is prepared by dropping PS nanoparticle suspension onto the electrochemically pretreated glassy carbon electrode surfaces (after the pretreatment process, the electrode surfaces will become more hydrophilic and suitable for the self-assembly of PS nanobeads) with a micropipette. The electrodes are then placed vertically and covered with a beaker, and the water is allowed to evaporate slowly. Electrodes coated with PS templates will be immersed into an aqueous solution containing pyrrole and respective drug (including model drug molecule like the fluorescent dyes) and electropolymerization will be carried out at a constant potential of +0.9 V. PPy loaded with drug will be formed within the interstitial spaces between the closely packed PS nanobeads. In order to obtain nanoporous PPy films, the PS templates can be removed by soaking in toluene for 12 hours. Then the electrodes are thoroughly rinsed with ethanol and water, respectively. Due to the inherent electrode-selective nature of electropolymerization, polypyrrole nanoporus film will be formed on the microelectrodes.

ii. Nanofibrous. Nanofibrous PPy can be electrochemically synthesized using nanoporous anodized aluminum oxide (AAO) membranes. A gold film is first sputtered on one side of the membrane to serve as a working electrode. The membrane is then attached to the carbon electrodes. PPy is electropolymerized in a drug containing solution at +0.9 V(vs. a Ag/AgCl reference). The length of the PPy nanofiber can be adjust by controlling the electropolymerization time. The resulting PPy nanofibers will be repeatedly washed with water. The highly pure Al film (several to tens of um thick) will be deposited on the microelectrodes by radio frequency sputtering. The Al film will then be anodized in sulfuric acid solution at 20 V to form nanopores. The resulting aluminum film is then etched away in 0.4 M H₃PO₄, 0.2 M H₂Cr₂O₄, and the remaining aluminum re-anodized under the same conditions until the Al film is fully oxidized. After the removal of the barrier layer through continuous anodizing, AAO membrane with nanopores is formed. Electropolymerization of pyrrole is carried out to synthesize nanofibers of PPy through the nanopores.

iii. Nanodrug Reservoirs. We have previously demonstrated that another convenient way to nanoengineer the conducting polymer film is to introduce nanotubes or nanoparticles as dopants. Carbon nanotubes and graphene oxide nansheets were shown to each load an impressive and tunable amount of drug molecules (Luo, et al., Biomaterials 32, 6316-6323 (2011); Luo et al., Journal of Materials Chemistry B 1, 1340-1348 (2013), each of which is hereby incorporated by reference in its entirety) and these drugs can be electrically released. The addition of these nanoreservoir also significantly increases the drug load while broadening the selection of drug molecules to include molecules of all charges (positive, negative and zwitterionic). To synthesize the film, multifunctional carbon nanotubes are first functionalized with carboxylic acid groups to render solubility and negative charges. Drug molecules are then incubated with the CNTs and strong sonication is used to load the drug inside and on the outer wall of the CNTs. Then the drug loaded CNTs are copolymerized with 3,4-ethylenedioxythiophene (EDOT) monomers and drug to form the drug loaded film on the carbon fiber tip. The detailed procedures can be found in Kolarcik et al. (Journal of Neural Engineering. 2015, 12(1). 016008, which is hereby incorporated by reference in its entirety).

### Example 14

### Characterization of Specific Molecule Uptake

This example provides methods for characterizing the uptake of a specific molecule. The drug uptake properties of the prepared nano-structured molecularly imprinted PPy films are characterized in two aspects: 1) The amount of drug they can uptake and 2) the specificity of the molecule uptake. The amount of drug uptake is characterized by dipping a synthesized molecularly imprinted PPy film into a target drug solution for a certain period of time to uptake the drug molecules, and the amount of uptaken drug can be calculated by measuring the concentration difference between the drug solution before and after the drug uptake. The specificity of the molecule uptake is measured by subjecting the molecularly imprinted PPy film to a mixture of molecules in various applications. The film is to selectively pick up the target molecule. For example, film prepared with GABA as template molecule is expected to uptake GABA selectively, and this can be tested by dipping the PPy film into a solution containing both GABA and Gamma-hydroxybutyric acid(GHBA), a molecule having very similar molecular structure. Both molecules will have the same concentration in the solution. By measuring the concentration differences of these two drugs before and after the uptake, the ratio of the GABA uptake/GHBA uptake can be obtained. High ratio will indicate the high specificity of the uptake wherein an effective specificity is the amount for inducing a biological response with a specific drug at the target location.

Uptake can also be measured using Electrochemical Quartz Crystal Microbalance (QCM). Here, polymer-drug film is synthesized on the EQCM crystal electrode. The electrode is soaked in the L and D GABA and dopamine respectively and the uptake can be directly measured by the mass increase on the crystal indicated by the resonance frequency change. In this case, a mixed drug solution is not used to test the specificity since the microbalance won't be able to differentiate different types of molecules. However, the ratio of uptake is still a good measure of the specificity.

Additionally, the uptake of fluorescent dye molecules from the coatings on microelectrodes can be additionally characterized using the time lapse imaging, which illustrates the real time response to uptake stimulus. In this case, the electrode will not be soaked in the dye solution to record the uptake because it will be very difficult to detect any uptake due to the high background intensity. Instead, we will image the dye release and apply uptake stimulus right after the dye release. The uptake of the released molecule will be picked up by the intensity reduction around the electrodes. This is a situation more closely resembles situations where one would need to apply uptake stimulus shortly after release stimulus to recycle the unused drugs. This system allows one to find the best parameters regarding the timing of two stimuli to maximize the lifetime of the release system.

Additional characterizations can be performed including those described elsewhere herein. For example, film micro and nano morphology can be monitored using SEM and AFM before and after the processing conditions and after the uptake. The surface area and porosity of the film is correlated to the drug uptake properties, so will the change of these parameters before and after treatment. To ensure the recording ability of the film, the impedance is monitored before and after each release. The electrical, chemical and mechanical stability characterization as those described herein can also be performed.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A wireless stimulation electrode, comprising:
a body made of biocompatible photovoltaic and/or photothermal material;
wherein at least one critical dimension of the body is substantially smaller than a target cell.

2. The wireless stimulation electrode of claim 1, further comprising at least one coating surrounding at least a portion of the body.

3. The wireless stimulation electrode of claim 2, wherein the at least one coating is functionalized with a bioactive material.

4. The wireless stimulation electrode of claim 2, wherein the at least one coating comprises a biocompatible dielectric coating and/or an optically selective material.

5. The wireless stimulation electrode of claim 2, wherein the at least one coating comprises a pharmacological agent which can be released upon photovoltaic and/or photothermal excitation.

6. The wireless stimulation electrode of claim 5, wherein the at least one coating contains a molecularly imprinted cavity that can be recharged by scavenging pharmacological agents or naturally produced molecules from the environment .

7. The wireless stimulation electrode of claim 1, further comprising one or more surface textures integral to the body, wherein the one or more surface textures are smaller than 1.5 microns.

8. The wireless stimulation electrode of claim 1, wherein the body comprises one or more of doped carbon nanotube, doped carbon fiber, doped graphene, or doped silicon.

9. The wireless stimulation electrode of claim 1, wherein the body has at least one critical dimension between 0.001-25 microns.

10. The wireless stimulation electrode of claim 1, wherein the photovoltaic material is configured to produce a voltage proportional to incident photons with wavelengths between 400-2,000 nanometers.

11. The wireless stimulation electrode of claim 1, further comprising:
a proximal portion;
a distal portion; and
an integrated micro-waveguide coupled to the proximal portion;
wherein the distal portion extends from the integrated micro-waveguide for stimulating a target cell.

12. A method for stimulating excitable tissue, comprising:
introducing at least one biocompatible photovoltaic and/or photothermal device into target excitable tissue; and
focusing photons, biphotons or multi-photons with wavelength between 400-2,000 nanometers onto the least one photovoltaic and/or photothermal device;
wherein the photovoltaic and/or photothermal device has one critical dimension smaller than a cell of the target excitable tissue.

13. The method of claim 12, further comprising introducing at least one micro-waveguide in the proximity of the at least one photovoltaic and/or photothermal device;
wherein the at least on micro-waveguide is configured to transmit photons, biphotons or multi-photons with wavelength between 400-2,000 nanometers.

14. The method of claim 13, wherein the focusing comprises deflecting the at least one micro-waveguide with at least one piezoelectric micromotor.

15. The method of claim 13, wherein the photovoltaic and/or photothermal device comprises an integrated micro-waveguide; and
wherein the introduced at least one micro-waveguide is adapted to focus photons, biphotons or multi-photons onto the integrated micro-waveguide.

16. The method of claim 13, further comprising:
introducing at least two biocompatible photovoltaic and/or photothermal devices into target excitable tissue;
wherein the at least two biocompatible photovoltaic and/or photothermal devices respond to different wavelengths of photons, multi-photons, or biphotons.

17. A system for wirelessly stimulating a neuron, comprising:
a sub-cellular sized biocompatible photovoltaic and/or photothermal device for stimulating excitable tissue; and
a coherent light source wirelessly coupled to the sub-cellular sized photovoltaic and/or photothermal device for stimulating excitable tissue.

18. The system of claim 17, wherein the subcellular sized photovoltaic and/or photothermal device and the coherent light source are physically separated.

19. The system of claim 17, wherein the coherent light source emits laser light in wavelengths between 400-2,000 nanometers.

20. The system of claim 17, wherein the coherent light source is an ultrafast coherent laser.

21. The system of claim 17, wherein the coherent light source is a vertical cavity surface emitting laser.

22. The system of claim 17, further comprising a focusing device adapted to focus light from the coherent light source onto the sub-cellular sized photovoltaic and/or photothermal device, thereby providing a system for wirelessly stimulating a neuron.

23. The system of claim 22, wherein the focusing device comprises a micro-waveguide and a piezoelectric micromotor.

24. A method of making a wireless stimulation electrode, comprising:
microfabricating a biocompatible photovoltaic and/or photothermal device with minimal photogalvanic and photoelectrochemical properties;
coating at least a portion of the photovoltaic and/or photothermal device with an optically selective material; and
coating at least a portion of the photovoltaic and/or photothermal device with a biocompatible insulating material;
wherein the dimension of the photovoltaic and/or photothermal device is between 0.01-25 microns.

25. The method of claim 24, wherein the biocompatible photovoltaic and/or photothermal device comprises one of doped carbon nanotube and doped silicon.

26. The method of claim 24, wherein the biocompatible insulating material and optically selective material have a combined thickness of less than 25 microns.

27. The method of claim 26, wherein the combined thickness is 0.8-5 microns.

28. The method of claim 24, wherein the optically selective coating allows selected wavelengths of light within the range 400-2,000 nanometers to pass.

29. The method of claim 24, wherein the biocompatible insulating material or optically selective coating is covalently functionalized with a bioactive material.

30. The method of claim 26, wherein the bioactive material comprises L1 cell adhesion molecule.

31. The method of claim 24, further comprising fabricating surface textures onto the photovoltaic and/or photothermal device, wherein the surface textures are less than 1,000 nm in diameter.
